SEQUENCE LISTING (30 sequences)

SEQ ID NO: 1 - DNA, 786 bp, Mycobacterium tuberculosis
atgcccaagagaagcgaatacaggcaaggcacgccgaactgggtcgaccttcagaccaccgatcagtccgccgccaaaaagttctacacatcgttgttcggctggggttacgacgacaacccggtccccggaggcggtggggtctattccatggccacgctgaacggcgaagccgtggccgccatcgcaccgatgcccccgggtgcaccggaggggatgccgccgatctggaacacctatatcgcggtggacgacgtcgatgcggtggtggacaaggtggtgcccgggggcgggcaggtgatgatgccggccttcgacatcggcgatgccggccggatgtcgttcatcaccgatccgaccggcgctgccgtgggcctatggcaggccaatcggcacatcggagcgacgttggtcaacgagacgggcacgctcatctggaacgaactgctcacggacaagccggatttggcgctagcgttctacgaggctgtggttggcctcacccactcgagcatggagatagctgcgggccagaactatcgggtgctcaaggccggcgacgcggaagtcggcggctgtatggaaccgccgatgcccggcgtgccgaatcattggcacgtctactttgcggtggatgacgccgacgccacggcggccaaagccgccgcagcgggcggccaggtcattgcggaaccggctgacattccgtcggtgggccggttcgccgtgttgtccgatccgcagggcgcgatcttcagtgtgttgaagcccgcaccgcagcaatag

SEQ ID NO: 2 - protein, 261 aa, Mycobacterium tuberculosis
MPKRSEYRQGTPNWVDLQTTDQSAAKKFYTSLFGWGYDDNPVPGGGGVYSMATLNGEAVAAIAPMPPGAPEGMPPIWNTYIAVDDVDAVVDKVVPGGGQVMMPAFDIGDAGRMSFITDPTGAAVGLWQANRHIGATLVNETGTLIWNELLTDKPDLALAFYEAVVGLTHSSMEIAAGQNYRVLKAGDAEVGGCMEPPMPGVPNHWHVYFAVDDADATAAKAAAAGGQVIAEPADIPSVGRFAVLSDPQGAIFSVLKPAPQQ

SEQ ID NO: 3 - DNA, 33 bp, Artificial Sequence
  Description of Artificial Sequence  Primer
attaaagcttacccgaccgcgtgaccagcggtc

SEQ ID NO: 4 - DNA, 33 bp, Artificial Sequence
  Description of Artificial Sequence  Primer
attatctagagatcatcctttcgttaggtggcg

SEQ ID NO: 5 - DNA, 32 bp, Artificial Sequence
  Description of Artificial Sequence  Primer
attatctagacagcaatagggagcatcccggg

SEQ ID NO: 6 - DNA, 26 bp, Artificial Sequence
  Description of Artificial Sequence  Primer
attagcagcgacggtgtcaacggttc

SEQ ID NO: 7 - DNA, 30 bp, Artificial Sequence
  Description of Artificial Sequence  Primer
attatctagaccctgtgaatagaggtccgc

SEQ ID NO: 8 - DNA, 30 bp, Artificial Sequence
  Description of Artificial Sequence  Primer
atttctagactggaggagatgatcgaggat

SEQ ID NO: 9 - DNA, 27 bp, Artificial Sequence
  Description of Artificial Sequence  Primer
atattaattaagatgcccaagagaagc

SEQ ID NO: 10 - DNA, 26 bp, Artificial Sequence
  Description of Artificial Sequence  Primer
attggatccctattgctgcggtgcgg

SEQ ID NO: 11 - DNA, 33 bp, Artificial Sequence
  Description of Artificial Sequence  Primer
ggactagtgcgatcagccagtcgatcagcagag

SEQ ID NO: 12 - DNA, 34 bp, Artificial Sequence
  Description of Artificial Sequence  Primer
ccttaattaattccgtcacagaatgctcctttac

SEQ ID NO: 13 - DNA, 28 bp, Artificial Sequence
  Description of Artificial Sequence  Primer
cccttaaatgtccgccacctaacgaaag

SEQ ID NO: 14 - DNA, 24 bp, Artificial Sequence
  Description of Artificial Sequence  Primer
cccaagcttctagcattctccgaa

SEQ ID NO: 15 - DNA, 25 bp, Artificial Sequence
  Description of Artificial Sequence  Primer
acggtgggtactaggtgtgggtttc

SEQ ID NO: 16 - DNA, 28 bp, Artificial Sequence
  Description of Artificial Sequence  Primer
tctgcgattactagcgactccgacttca

SEQ ID NO: 17 - DNA, 18 bp, Artificial Sequence
  Description of Artificial Sequence  Primer
ggcgatctggtgggcccg

SEQ ID NO: 18 - DNA, 21 bp, Artificial Sequence
  Description of Artificial Sequence  Primer
cgccggaggcattagcacgct

SEQ ID NO: 19 - DNA, 19 bp, Artificial Sequence
  Description of Artificial Sequence  Primer
acgcgaagtcgggcaacac

SEQ ID NO: 20 - DNA, 22 bp, Artificial Sequence
  Description of Artificial Sequence  Primer
gcggcagcgggcgggagcaact

SEQ ID NO: 21 - DNA, 27 bp, Artificial Sequence
  Description of Artificial Sequence  Primer
atgcccaagagaagcgaatacaggcaa

SEQ ID NO: 22 - DNA, 24 bp, Artificial Sequence
  Description of Artificial Sequence  Primer
ctattgctgcggtgcgggcttcaa

SEQ ID NO: 23 - DNA, 24 bp, Artificial Sequence
  Description of Artificial Sequence  Primer
cgaggggattacacatgaccaact

SEQ ID NO: 24 - DNA, 19 bp, Artificial Sequence
  Description of Artificial Sequence  Primer
cggaagagcgcccaatacg

SEQ ID NO: 25 - DNA, 24 bp, Artificial Sequence
  Description of Artificial Sequence  Primer
ataacgttctcggctcgatgatcc

SEQ ID NO: 26 - DNA, 29 bp, Artificial Sequence
  Description of Artificial Sequence  Primer
atcccctgattctgtggataaccgtatta

SEQ ID NO: 27 - DNA, 22 bp, Artificial Sequence
  Description of Artificial Sequence  Primer
accaccttgtccaccaccgcat

SEQ ID NO: 28 - DNA, 24 bp, Artificial Sequence
  Description of Artificial Sequence  Primer
cgaatcattggcacgtctactttg

SEQ ID NO: 29 - DNA, 23 bp, Artificial Sequence
  Description of Artificial Sequence  Primer
gtggcttggcgggcacggtggag

SEQ ID NO: 30 - DNA, 25 bp, Artificial Sequence
  Description of Artificial Sequence  Primer
gtggcaccggcggcaccgcacacct

(19) United States
(12) Patent Application Publication
(10) Pub. No.: US 2003/0199012 A1
(43) Pub. Date: Oct. 23, 2003

(54) Compositions and methods for treatment of infectious and inflammatory diseases

(76) Inventor: John L. Ho, New York, NY (US)

Correspondence Address: Michael L. Goldman, Esq., NIXON & PEABODY LLP, Clinton Square, P.O. Box 31051, Rochester, NY, 14603-1051, US

(21) Appl. No.: 10/357,043

(22) Filed: Jan. 31, 2003

Related U.S. Application Data
(60) Provisional application No. 60/353,985, filed on Feb. 1, 2002.

(51) Int. Cl. A61K 39/40; G01N 33/554; G01N 33/569; C07H 21/04; C12P 21/02; C12N 1/21; C07K 14/35; C07K 16/12

(52) U.S. Cl. 435/7.32; 424/168.1; 435/69.3; 435/252.3; 435/320.1; 530/350; 536/23.7; 530/388.4

(57) ABSTRACT

The present invention relates to a nucleic acid construct having a nucleic acid molecule that encodes a factor suppressing an immune response to Mycobacterium tuberculosis in a host subject; an isolated antibody against the protein or polypeptide encoded by the nucleic acid molecule; and uses for the protein and its antibody, including in a method for detection of Mycobacterium tuberculosis in a sample of tissue or body fluids; a method of vaccinating a mammal against infection by Mycobacterium tuberculosis; a vaccine for preventing infection and disease of mammals by Mycobacterium tuberculosis and for actively immunizing mammals against Mycobacterium tuberculosis; and methods of treating inflammatory disease in mammals.

This application claims the benefit of U.S. Provisional Patent Application Serial No. 60/353,985, filed Feb. 1, 2002.

The subject matter of this application was made with support from the United States Government under the National Institutes of Health Grant Nos. AM39606, HL61960, and TW00018. The U.S. Government may have certain rights.

### FIELD OF THE INVENTION

The present invention relates to compositions and methods for the detection, treatment, and prevention of Mycobacterium tuberculosis infection.

### BACKGROUND OF THE INVENTION

Control of Mycobacterium tuberculosis (Mtb), the etiologic agent of tuberculosis (Th), is immune cell mediated as shown by humans without a functioning interferon gamma receptor (IFN-λR) or interleukin-12 receptor (IL-12R) manifesting with disseminated mycobacteria disease (Dorman et al., “Interferon-Gamma and Interleukin-12 Pathway Defects and Human Disease,” Cytokine Growth Factor Rev. 11(4):321-33 (2000); Jouanguy et al., “IL-12 and IFN-Gamma in Host Defense Against Mycobacteria and Salmonella in Mice and Men,”Curr. Opin. Immunol. 11(3):346-51 (1999); Altare et al., “Inherited Interleukin 12 Deficiency in a Child with Bacille Calmette-Guerin and Salmonella Enteritidis Disseminated Infection,” J. Clin. Invest. 102(12):2035-40 (1998); Sakai et al., “Missense Mutation of the Interleukin-12 Receptor Beta 1 Chain-Encoding Gene is Associated with Impaired Immunity Against Mycobacterium avium Complex Infection,” Blood 97(9):2688-94 (2001)). Moreover, immunosuppression by drugs, cancer, HIV-1 or immune senescence is associated with reactivation Tb, highlighting the fact that Mtb avoids immune elimination to establish life-long infection (Rook et al., “Advances in the Immunopathogenesis of Pulmonary Tuberculosis,” Curr. Opin. Pulm. Med. 7(3):1 16-23 (2001); Flynn et al., “Immunology of Tuberculosis,” Annu. Rev. Immunol. 19:93-129 (2001); Ho et al., “Defenses of the Lung Against Tuberculosis,” in The Lung: Scientific Foundations, Crystal et al., eds., 2nd Edition, Chapter 183, pp. 2381-94 (1997); Vanham et al., “Examining a Paradox in the Pathogenesis of Human Pulmonary Tuberculosis: Immune Activation and Suppression/Anergy,” Tuber. Lung Dis. 78(3-4):145-58 (1997); Ellner, “Regulation of the Human Immune Response During Tuberculosis,” J. Lab. Clin. Med. 130(5):469-75 (1997)). This accounts for one in three persons worldwide having latent Mtb infection and a 5-10% lifetime risk of progression to active disease, translating to ˜8 million annual active Tb cases and ˜3 million annual deaths (Bishai, “The Mycobacterium tuberculosis Genomic Sequence: Anatomy of a Master Adaptor,” Trends Microbiol. 6(12):464-5 (1998)). Genes present in Mtb but absent in non-pathogenic mycobacteria are proposed as virulence factors. However, which Mtb specific genes mediate rapid progression to disease or transit to latent infection, and how these genes function, remain poorly defined.

There are several examples whereby specific gene products of microbes modulate the host immune response to effect microbial survival (Orth et al., “Disruption of Signaling by Yersinia Effector YopJ, a Ubiquitin-Like Protein Protease,” Science 290(5496):1594-7 (2000); Boland et al., “Role of YopP in Suppression of Tumor Necrosis Factor Alpha Release by Macrophages During Yersinia Infection,” Infect. Immun. 66(5):1878-84 (1998); Cornelis et al., “Yersinia Lead SUMO Attack,” Nat. Med. 7:21-23 (2001); Trufariello et al., “Adenovirus E3 14.7-kDa Protein, an Antagonist of Tumor Necrosis Factor Cytolysis, Increases the Virulence of Vaccinia Virus in Severe Combined Immunodeficient Mice,” Proc. Natl. Acad. Sci. USA 91:10987-91 (1994); Trufariello et al., “Adenovirus E3 14.7-kDa Protein, an Antagonist of Tumor Necrosis Factor Cytolysis, Increases the Virulence of Vaccinia Virus in a Murine Pneumonia Model,” J. Virol. 68:453-62 (1994); Nigou et al., “Mannosylated Lipoarabinomannans Inhibit IL-12 Production by Human Dendritic Cells: Evidence for a Negative Signal Delivered through the Mannose Receptor,” J. Immunol. 166(12):7477-85 (2001); Stockl et al., “Human Major Group Rhinoviruses Down-Modulate the Accessory Function of Monocytes by Inducing IL-10,” J. Clin. Invest. 104(7):957-65 (1999); Fleming et al., “A Homolog of Interleukin-10 is Encoded by the Poxvirus Orf Virus,” J. Virol. 71(6):4857-61 (1997); Vockerodt et al., “The Epstein-Barr Virus Latent Membrane Protein 1 Induces Interleukin-10 in Burkitt's Lymphoma Cells but not in Hodgkin's Cells Involving the p38/SAPK2 Pathway,” Virology 280(2):183-98 (2001); Henke et al., “Viral IL-10 Gene Transfer Decreases Inflammation and Cell Adhesion Molecule Expression in a Rat Model of Venous Thrombosis,” J. Immunol. 164(4):2131-41 (2000); Suzuki et al., “Viral Interleukin 10 (IL-10), the Human Herpes Virus 4 Cellular IL-10 Homologue, Induces Local Anergy to Allogeneic and Syngeneic Tumors,” J. Exp. Med. 182(2):477-86 (1995); Wynn et al., “Analysis of Granuloma Formation (by Schistomsoma eggs) in Double Cytokine-Deficient Mice Reveals a Central Role for IL-10 in Polarizing Both T Helper Cell 1- and T Helper Cell 2-Type Cytokine Responses In vivo,” J. Immunol. 159(10):5014-23 (1997); Barcova et al., “gp41 Envelope Protein of Human Immunodeficiency Virus Induces Interleukin (IL)-10 in Monocytes, but not in B, T, or NK Cells, Leading to Reduced IL-2 and Interferon-Gamma Production,” J. Infect. Dis. 177(4):905-13 (1998); Taoufik et al., “Human Immunodeficiency Virus gp120 Inhibits Interleukin-12 Secretion by Human Monocytes: an Indirect Interleukin-10-Mediated Effect,” Blood 89(8):2842-8 (1997); Koutsonikolis et al., “HIV-1 Recombinant gp41 Induces IL-10 Expression and Production in Peripheral Blood Monocytes but not in T-Lymphocytes,” Immunol. Lett. 55(2):109-13 (1997); Schols et al., “Human Immunodeficiency Virus Type 1 gp120 Induces Anergy in Human Peripheral Blood Lymphocytes by Inducing Interleukin-10 Production,” J. Virol. 70(8):4953-60 (1996)). Specifically, Epstein-Barr virus (EBV) encodes a human IL-10 homolog as well as the EBV latent protein-1 that induces IL-10 (Vockerodt et al., “The Epstein-Barr Virus Latent Membrane Protein 1 Induces Interleukin-10 in Burkitt's Lymphoma Cells but not in Hodgkin's Cells Involving the p38/SAPK2 Pathway,” Virology 280(2):183-98 (2001); Henke et al., “Viral IL-10 Gene Transfer Decreases Inflammation and Cell Adhesion Molecule Expression in a Rat Model of Venous Thrombosis,” J. Immunol. 164(4):2131-41 (2000); Suzuki et al., “Viral Interleukin 10 (IL-10), the Human Herpes Virus 4 Cellular IL-10 Homologue, Induces Local Anergy to Allogeneic and Syngeneic Tumors,” J. Exp. Med. 182(2):477-86 (1995)). Both of these EBV factors are thought to facilitate viral survival and pathogenesis through IL-10's immune suppressive activity (Vockerodt et al., “The Epstein-Barr Virus Latent Membrane Protein 1 Induces Interleukin-10 in Burkitt's Lymphoma Cells but not in Hodgkin's Cells Involving the p38/SAPK2 Pathway,” Virology 280(2):183-98 (2001); Henke et al., “Viral IL-10 Gene Transfer Decreases Inflammation and Cell Adhesion Molecule Expression in a Rat Model of Venous Thrombosis,” J. Immunol. 164(4):2131-41 (2000); Suzuki et al., “Viral Interleukin 10 (IL-10), the Human Herpes Virus 4 Cellular IL-10 Homologue, Induces Local Anergy to Allogeneic and Syngeneic Tumors,” J. Exp. Med. 182(2):477-86 (1995)). IL-10 is a potent inhibitor of inflammatory response to pathogens, suppressing the production of cytokines such as tumor necrosis factor alpha (TNF-α), IL-12, IFN-λ and expression of macrophage NOS2 as well as costimulatory molecules such as CD40, CD80, and CD86, immune factors involved in control of Mtb infection (Brossart et al., “Tumor Necrosis Factor-α and CD40 Ligand Antagonize the Inhibitory Effects of Interleukin 10 and T-Cell Stimulatory Capacity of Dendritic Cess l.,” Can. Res. 60:4485-92 (2000); Gao et al., “CD40-Deficient Dendritic Cells Producing Interleukin-10, but not Interleukin-12, Induce T-cell Hyporesponsiveness In vitro and Prevent Acute Allograft Rejection,” Immunology 98(2):159-70 (1999); Villegas et al., “Blockade of Costimulation Prevents Infection-Induced Immunopathology in IL-10-Deficient Mice,” Infect. Immun. 68:2837-44 (2000); Van Gool et al., “Blocking CD40-CD154 and CD80/CD86-CD28 Interactions During Primary Allogeneic Stimulation Results in T Cell Anergy and High IL-10 Production,” Eur. J. Immunol. 29(8):2367-75 (1999); Akdis et al., “Mechanisms of Interleukin-10-Mediated Immune Suppression,” Immunology 103(2):131-6 (2001)). In addition, there is growing evidence that IL-10 is involved in Tb. Specifically, IL-10 plays a critical role in murine model of M. bovis Bacillus Calmitte-Guerin (BCG) infection, because IL-10 over-expression enhanced bacilli growth while IL-10 depletion by gene knock-out (KO) increased anti-mycobacterial immunity and lowered BCG load (Murray et al., “Increased Antimycobacterial Immunity in Interleukin-10-Deficient Mice,” Infect. Immun. 67(6):3087-95 (1999); Murray et al., “T Cell-Derived IL-10 Antagonizes Macrophage Function in Mycobacterial Infection,” J. Immunol. 158(l):315-21 (1997); Jacobs et al., “Increased Resistance to Mycobacterial Infection in the Absence of Interleukin-10,” Immunology 100(4):494-501(2000)). Clinical data also lend support for IL-10 in Tb pathogenesis, because neutralization of IL-10 from peripheral blood cells from active Tb patients enhanced Mtb specific T cell proliferation and IFN-λ production and increased monocyte production of IL-12 and CTLA-4 expression (Samten et al., “Depressed CD40 Ligand Expression Contributes to Reduced Gamma Interferon Production in Human Tuberculosis,” Infect. Immun. 68(5):3002-6 (2000); Gong et al., “Interleukin-10 Downregulates Mycobacterium tuberculosis-Induced Th1 Responses and CTLA-4 Expression,” Infect. Immun. 64(3):913-8 (1996)), IL-10 mediates the anergy seen in some patients with active Tb (Baliko et al., “Th2 Biased Immune Response in Cases with Active Mycobacterium tuberculosis Infection and Tuberculin Anergy,” FEMS Immunol. Med. Microbiol. 22(3):199-204 (1998); Boussiotis et al., “IL-10-Producing T Cells Suppress Immune Responses in Anergic Tuberculosis Patients,” J. Clin. Invest. 105(9):1317-25 (2000)), predominant T cell clones obtained from the lungs of active Tb cases secrete both IL-10 and IFN-λ (Rook et al., “Advances in the Immunopathogenesis of Pulmonary Tuberculosis,” Curr. Opin. Puhm. Med. 7(3):116-23 (2001); Ho et al., “Defenses of the Lung Against Tuberculosis,” in The Lung: Scientific Foundations, Crystal et al., eds., 2nd Edition, Chapter 183, pp. 2381-94 (1997); McAdam et al., “Polarization of PPD-Specific T-Cell Response of Patients with Tuberculosis from Th0 to Th1 Profile After Successful Antimycobacterial Therapy or In vitro Conditioning with Interferon-Alpha or Interleukin-12,” Am. J. Respir. Cell Mol. Biol. 24(2):187-94 (2001)), and IL-10 production is triggered by Mtb infection (Gong et al., “Interleukin-10 Downregulates Mycobacterium tuberculosis-Induced Th1 Responses and CTLA-4 Expression,” Infect. Immun. 64(3):913-8 (1996); Almeida et al., “Induction of In vitro Human Macrophage Anti-Mycobacterium tuberculosis Activity: Requirement for Interferon-λ and Primed-Lymphocytes,” J. Immunol. 160:4490-9 (1998); Fulton et al., “Regulation of Interleukin-12 by Interleukin-10, Transforming Growth Factor-Beta, Tumor Necrosis Factor-Alpha, and Interferon-Gamma in Human Monocytes Infected with Mycobacterium tuberculosis H37Ra,” J. Infect. Dis. 178(4):1105-14 (1998); Giacomini et al., “Infection of Human Macrophages and Dendritic Cells with Mycobacterium tuberculosis Induces a Differential Cytokine Gene Expression That Modulates T Cell Response,” J. Immunol. 166(12):7033-41 (2001)).

Macrophages are the preferred cell for intracellular survival of Mtb. It is also recognized that the macrophage and Mtb interaction may be critical to the outcome of infection by Mtb. This is underscored by the finding that depletion of alveolar macrophages in mice exerted protective effects for pulmonary Tb (Leemans et al., “Depletion of Alveolar Macrophages Exerts Protective Effects in Pulmonary Tuberculosis in Mice,” J. Immunol. 166(7):4604-11 (2001)) and patients with silicosis (where lung macrophages are paralyzed by the inhaled silicate) have an increased risk for active Tb (Davies, “Silicosis and Tuberculosis Among South African Goldminers—An Overview of Recent Studies and Current Issues,” S. Afr. Med. J. 91(7):562-6 (2001) Review)). In addition, murine models of Tb have shown that susceptible mice (Balb/C or I/St), in contrast to resistant mice (C56B16 or A/Sn), produced higher amounts of IL-10, lower amounts of IFN-, and IL-12, and their macrophages expressed lower NOS2, thereby contributing to the severity of disease (Yoshida et al., “Dissection of Strain Difference in Acquired Protective Immunity Against Mycobacterium bovis Calmette-Guerin Bacillus (BCG). Macrophages Regulate the Susceptibility Through Cytokine Network and the Induction of Nitric Oxide Synthase,” J. Immunol. 155(4):2057-66 (1995)). Several groups have reported that in vitro Mtb infection of human monocyte/macrophages is associated with high IL-10 production. In addition, in a human cell culture model of immune control of MtbH37Ra infection, low IL-10 production was associated with reduction in bacilli load while high IL-10 was associated with uncontrolled growth of Mtb. What is needed now is clear evidence that the Rv0577 gene of Mtb is an immunomodulatory factor in Mtb infection, and methods which utilize this gene, and its protein product, for the detection, prevention, and treatment of Mycibacterium tuberculosis infection.

The present invention is directed to overcoming these and other deficiencies in the art.

### SUMMARY OF THE INVENTION

The present invention relates to a nucleic acid construct having a nucleic acid molecule that encodes a factor suppressing an immune response to Mycobacterium tuberculosis in a host subject, where the nucleic acid molecule either: 1) has a nucleotide sequence corresponding to SEQ ID NO: 1; 2) has a nucleotide sequence that hybridizes to the nucleic acid corresponding to SEQ ID NO: 1 under stringent conditions characterized by a hybridization buffer comprising 5×SSC at a temperature of 54° C.; 3) is at least 55% similar to the nucleotide sequence of SEQ ID NO: 1 by basic BLAST using default parameters analysis; or 4) encodes a protein or polypeptide having an amino acid sequence corresponding to SEQ ID NO: 2; and has an operably linked DNA promoter and an operably linked 3′ regulatory region.

The present invention also relates to an isolated antibody, or binding portion thereof, against a protein or polypeptide having an amino acid corresponding to SEQ ID NO: 2.

Another aspect of the present invention is a method for detection of Mycobacterium tuberculosis specific antibodies in a sample of tissue or body fluids. This method involves providing an isolated protein or polypeptide having an amino acid corresponding to SEQ ID NO: 2 as an antigen; contacting the sample with the antigen under conditions effective to allow formation of a complex of the antigen bound to antibodies which recognize the antigen; and detecting if any of the complex is present, thereby indicating a presence of Mycobacterium tuberculosis the sample.

The present invention also relates to another method for detection of Mycobacterium tuberculosis in a sample of tissue or body fluids. This method involves providing an antibody or binding portion thereof against the protein or polypeptide of the present invention having an amino acid corresponding to SEQ ID NO: 2, contacting the sample with the antibody or binding portion thereof under conditions effective to allow formation of a complex of the antibody or binding portion thereof and an antigen recognized by the antibody or binding portion thereof, and detecting if any of the complex is present, thereby indicating a presence of Mycobacterium tuberculosis in the sample.

The present invention also relates to a third method for detection of Mycobacterium tuberculosis in a sample of tissue or body fluids. This method involves providing a nucleic acid molecule as a probe in a nucleic acid hybridization assay; contacting the sample with the probe under conditions effective to permit formation of a complex of the probe and nucleic acid which hybridizes to the probe; and detecting formation of the complex in the sample, thereby indicating a presence of Mycobacterium tuberculosis in the sample. The nucleic acid molecule either: 1) has a nucleotide sequence corresponding to SEQ ID NO: 1; 2) has a nucleotide sequence that hybridizes to the nucleic acid corresponding to SEQ ID NO: 1 under stringent conditions characterized by a hybridization buffer comprising 5×SSC at a temperature of 54° C.; 3) is at least 55% similar to the nucleotide sequence of SEQ ID NO: 1 by basic BLAST using default parameters analysis; or 4) encodes a protein or polypeptide having an amino acid sequence corresponding to SEQ ID NO: 2.

The present invention also relates to a fourth method of detection of Mycobacterium tuberculosis in a sample of tissue or body fluids. This method involves providing a nucleic acid molecule as a probe or primer in a gene amplification detection procedure, contacting the sample with the probe or primer under conditions effective to amplify probe or primer-specific nucleic acid molecules; and detecting any amplified probe or primer-specific molecules, thereby indicating a presence of Mycobacterium tuberculosis in the sample. The nucleic acid molecule either: 1) has a nucleotide sequence corresponding to SEQ ID NO: 1; 2) has a nucleotide sequence that hybridizes to the nucleic acid corresponding to SEQ ID NO: 1 under stringent conditions characterized by a hybridization buffer comprising 5×SSC at a temperature of 54° C.; 3) is at least 55% similar to the nucleotide sequence of SEQ ID NO: 1 by basic BLAST using default parameters analysis; or 4) encodes a protein or polypeptide having an amino acid sequence corresponding to SEQ ID NO: 2.

The present invention also relates to a method of vaccinating a mammal against infection by Mycobacterium tuberculosis. This method involves administering an effective amount of an isolated protein or polypeptide having an amino acid sequence corresponding to SEQ ID NO: 2 to the mammal.

Another aspect of the present invention is a vaccine for preventing infection and disease of mammals by Mycobacterium tuberculosis. This vaccine includes an isolated protein or polypeptide having an amino acid sequence corresponding to SEQ ID NO: 2; and a pharmaceutically-acceptable carrier.

Another aspect of the present invention is a method of vaccinating mammals against infection by Mycobacterium tuberculosis. This involves administering to mammals an effective amount of the vaccine of the present invention that includes an isolated protein or polypeptide having an amino acid sequence corresponding to SEQ ID NO: 2 and a pharmaceutically-acceptable carrier.

Another aspect of the present invention is a method of treating mammals infected with Mycobacterium tuberculosis. This method involves administering an effective amount of the isolated antibody, or binding portion thereof, against a protein or polypeptide having an amino acid corresponding to SEQ ID NO: 2, to mammals infected with Mycobacterium tuberculosis.

Another aspect of the present invention is a composition for passively immunizing mammals infected with Mycobacterium tuberculosis. This composition includes an isolated antibody, or binding portion thereof, against a protein or polypeptide having an amino acid corresponding to SEQ ID NO: 2, and a pharmaceutically-acceptable carrier.

Another aspect of the present invention is a method for passively immunizing mammals infected with Mycobacterium tuberculosis. This method involves administering an effective amount of the composition of the present invention having an isolated antibody, or binding portion thereof, against a protein or polypeptide having an amino acid corresponding to SEQ ID NO: 2, and a pharmaceutically-acceptable carrier.

Another aspect of the present invention relates to a method of enhancing vaccination against Mycobacterium tuberculosis using a composition comprising a microorganism capable of producing an antigenic response against Mycobacterium tuberculosis when introduced into a host subject. This method involves suppressing in the microorganism the expression of a nucleic acid molecule that either: 1) has a nucleotide sequence corresponding to SEQ ID NO: 1; 2) has a nucleotide sequence that hybridizes to the nucleic acid corresponding to SEQ ID NO: 1 under stringent conditions characterized by a hybridization buffer comprising 5×SSC at a temperature of 54° C.; 3) is at least 55% similar to the nucleotide sequence of SEQ ID NO: 1 by basic BLAST using default parameters analysis; or 4) encodes a protein or polypeptide having an amino acid sequence corresponding to SEQ ID NO: 2.

The present invention also relates to a composition for actively immunizing mammals against Mycobacterium tuberculosis. This composition has a microorganism capable of producing an antigenic response against Mycobacterium tuberculosis when introduced into a host subject, where the microorganism has been modified to be incapable of producing a nucleic acid molecule encoding a factor suppressing an immune response to Mycobacterium tuberculosis in a host, and a pharmaceutically-acceptable carrier.

Another aspect of the present invention relates to a method of vaccinating a mammal against infection by Mycobacterium tuberculosis. This method involves administering an effective amount of a composition having a microorganism capable of producing an antigenic response against Mycobacterium tuberculosis, where the microorganism has been modified to be incapable of producing a nucleic acid molecule encoding a factor suppressing an immune response to Mycobacterium tuberculosis in a host, and a pharmaceutically-acceptable carrier.

Another aspect of the present invention is a method of treating inflammatory disease in a mammal. This method involves providing a nucleic acid construct having a nucleic acid molecule that encodes a factor suppressing an immune response to Mycobacterium, where the nucleic acid molecule either: 1) has a nucleotide sequence corresponding to SEQ ID NO: 1; 2) has a nucleotide sequence that hybridizes to the nucleic acid corresponding to SEQ ID NO: 1 under stringent conditions characterized by a hybridization buffer comprising 5×SSC at a temperature of 54° C.; 3) is at least 55% similar to the nucleotide sequence of SEQ ID NO: 1 by basic BLAST using default parameters analysis; or 4) encodes a protein or polypeptide having an amino acid sequence corresponding to SEQ ID NO: 2; and operably linked 5′ and 3′ regulatory elements. The nucleic acid construct is administered to a mammal under conditions effective to treat an inflammatory disease.

The present invention also relates to another method of treating inflammatory disease in a mammal. This method involves providing a protein or polypeptide that suppresses an immune response to Mycobacterium tuberculosis, where the protein or polypeptide has an amino acid sequence of SEQ ID NO: 2; and administering the protein or polypeptide to a mammal under conditions effective to treat an inflammatory disease.

### BRIEF DESCRIPTION OF THE DRAWINGS

FIG. 1 is a graph comparing IL-10 induction by M. tuberculosis and M. smegmatis. Induction of IL-10 by mycobacteria was performed using human peripheral blood monocytes from a leukocyte rich blood bank preparation purified by negative selection (up to 90% pure by CD14 expression on FACS analysis) and cultured in X-Vivo-20 medium (BioWhittiker, an artificial medium without protein or detectable endotoxin). The results shown are the mean i SD of M. smegmatis (Ms, ATCC No. 23038, lots 961, 972, or mc²155), Mtb H37Rv (MtbRv, ATCC 27294, lots 013, 082) or MtbH37Ra (MtbRa, ATCC 25177, lot 082), at 0.5 colony forming units (cfu) stimulation per monocyte at 10⁶ monocytes per well (in duplicate) from 5 to 8 separate donors, P<0.03 for MtbRv or MtbRa compared to Ms; P>0.05 for Ms versus medium, Students' paired t-test. LPS (E. coli lipopolysaccharide, 1 μg/ml).

FIGS. 2A-C are graphs showing IL-10 inducing activity by Mtb H37Rv preparations. FIG. 2A shows IL-10 production in cell-free supernatants from cultures of blood bank donor monocytes (1×10⁶/well, isolated by self-aggregation method) at 48 h after stimulation (most in triplicates) with Mtb H37Rv (0.5 cfu per monocyte) or with Mtb H37Rv components, cell wall (1 μg/ml), cytosol (1 μg/ml), membrane (1 μg/ml), or culture filtrate (CFP, 1 μg/ml) and purified protein derivative (PPD, 1 μg/ml) of Mtb. FIG. 2B shows the effect of varying doses of MtbH37Rv CFP on IL-10 production. FIG. 2C shows enrichment of IL-10 activity by anion exchange chromatography. Monocytes were stimulated with 0.2 μg/ml of each fraction (fx) of MtbH37Rv CFP produced by anion exchange (QAE) chromatography. CFP fractionated by anion exchange (QAE) chromatography. The results are the mean ±SD of indicated number (n) of donors.

FIGS. 3A-C are graphs showing the induction of cytokines by MtbH37Rv or preparations of Mtb. Cell-free supernatants obtained from blood bank donor monocytes (1×10⁶/well, self-aggregation method) at 48 h after stimulation (in triplicate) with MtbH37Rv mannose capped lipoarabinomannan (manLAM, 5 μg/ml), MtbH37Rv CFP (0.5 to 1 μg/ml) or MtbH37Rv CFP fx9 by anion exchange chromatography (0.2 μg/ml), or 0.5 cfu MtbH37Ra per monocytes. FIG. 3A shows IL-10 assay results. FIG. 3B shows TNF-α assay results. FIG. 3C shows assay results using IL-1β antibodies. All results are the mean ±SD of 8 to 14 donors tested with each Mtb reagent.

FIGS. 4A-E show the creation and growth of 577 null Mtb and a complemented 577 null mutant Mtb. FIG. 4A is a Southern blot of genomic DNA digested by PVUII, separated by electrophoresis, transferred to membrane and analyzed by Southern blot performed using a digoxitonin-labeled Rv0577 probe obtained by PCR with detection by chemiluminescence. FIG. 4B is a PCR amplification analysis using gene amplification primer pairs previously reported to detect all mycobacteria (16S rRNA), only Mtb complex subspecies (MPB70), only M. smegmatis (Ms0911), Rv0577 (cfp32), or the insertion of Rv0577 into the multiple cloning site of pMSG (MCS pMSG). FIG. 4C is a Western blot analysis of the parental MtbH37Rv, Rv0577 null mutant, and Rv0577 complemented null mutant. The Rv0577 null mutant was transformed with pMSG.577 plasmid in which Rv0577 expression is under the regulation of the constitutive Mtb glutamine synthase promoter. Cell lysates (1 μg) were separated by electrophoresis on Tris-Bis acrylamide, transferred to nitrocellulose, and recombinant Rv0577 or unknown sample were probed with rabbit polyclonal anti-rRv0577 antisera (3^{rd} bleed) and developed with anti-rabbit Ab linked to horseradish peroxidase chemiluminescent assay. FIG. 4D is a graph showing the growth kinetics of parental, 577 null mutant, and complementated 577 null mutant Mtb, quantified by OD580 nm of cultures inoculated into 7H9 broth supplemented with ADC (plus hygromycin B 50 μg/ml for 577 null mutant or plus kanamycin 25 μg/ml for complementated 577 null mutant). FIG. 4E shows the results of the broth cultures from FIG. 4D after being plated on 7H11 agar supplemented with OADC and antibiotics. Illustrated are the mean ±SD of three independent experiments. FIGS. 4F-H show the colony morphology of parental, 577 null mutant, and complementated 577 null mutant Mtb, respectively, grown on7H11 agar.

FIGS. 5A-B show the characterization of Rv0577. FIG. 5A shows IL-10 production resulting from a challenge by 577 null mutant compared with parental was statistically significant (n=7 separate donors, mean ±SE; P≦0.01, Students' paired t-test). FIG. 5B shows TNF-α production assayed using the same culture supernatants; P>0.05, mean±SD. Inducti6n of IL-10 by mycobacteria or LPS was performed using human peripheral blood monocytes (10⁶ per well in 1 ml) purified by negative selection and cultured in X-Vivo-20 medium. Parental, Rv0577 null mutant, Rv0577 complemented (pMSG.577), null mutant or laboratory assay standard Mtb H37Rv at 0.1 or 0.5 cfu per monocyte were compared with medium control or LPS (100 ng/ml) stimulation of monocyte production of IL-10 or TNF-α at 48 h.

FIGS. 6A-E show the results of over-expression of Rv0577 in M. smegmatis and the effect of Rv0577 overexpression on IL-10 and TNF-α production. FIG. 6A shows gene amplification by PCR analysis of parental M. smegmatis (Ms) and M. smegmatis transformants possessing pMS3.577 and pMS3 plasmids. The pMS3 plasmid contains the hygromycin resistance gene under the control of the constitutive M. smegmatis heat shock protein promoter. Gene amplification utilized primer pairs as described for FIG. 4B, supra, and primer pairs for the backbone of pMS3 in order to visualize a backbone DNA fragment or the backbone plus a DNA insert. FIG. 6B is a Western blot analysis of cell lysates (1 μg amounts) of the parental M. smegmatis and M. smegmatis transformants possessing pMS3.577 and pMS3 plasmids, studied for expression of Rv0577 protein as detailed in FIG. 4 legend, supra. FIG. 6C is a graph showing IL-10 production of M. smegmatis (Ms) infected human monocytes in comparison to MtbRvH37 infected and LPS-treated monocytes. In FIGS. 6D-E, M. smegmatis (strain MC²155) transformed with pMS3 plasmid or pMS3.577 were grown in 7H11 medium containing hygromycin 10 μg/ml, and washed bacilli were used to generate whole cell lysate or to infected monocytes. Human monocytes freshly isolated by negative selection were infected with M. smegmatis transformants containing pMS3 or pMS3.577 plasmid at 0.04 to 0.05 cfu to monocyte ratio. Cell supernatant were assayed for IL-10, shown in FIG. 6D, or TNF-α production, shown in FIG. 6E (mean ±SD; n=4, P≦0.01, using absolute IL-10 values between M. smegmatis transformants containing pMS3 or pMS3.577).

### DETAILED DESCRIPTION OF THE INVENTION

The present invention relates to a nucleic acid construct having a nucleic acid molecule that encodes a factor suppressing an immune response to Mycobacterium tuberculosis in a host subject, where the nucleic acid molecule either: 1) has a nucleotide sequence corresponding to SEQ ID NO: 1 herein; 2) has a nucleotide sequence that hybridizes to the nucleic acid corresponding to SEQ ID NO: 1 under stringent conditions characterized by a hybridization buffer comprising 5×SSC at a temperature of 54° C.; 3) is at least 55% similar to the nucleotide sequence of SEQ ID NO: 1 by basic BLAST using default parameters analysis; or 4) encodes a protein-or polypeptide having an amino acid sequence corresponding to SEQ ID NO: 2 herein, and has an operably linked DNA promoter and an operably linked 3′ regulatory region.

One isolated nucleotide sequence suitable as a nucleic acid molecule of the construct of the present invention has a nucleotide sequence of SEQ ID NO: 1, as follows:

| | |
|---|---|
| atgcccaaga gaagcgaata caggcaaggc acgccgaact gggtcgacct tcagaccacc | 60 |
| gatcagtccg ccgccaaaaa gttctacaca tcgttgttcg gctggggtta cgacgacaac | 120 |
| ccggtccccg gaggcggtgg ggtctattcc atggccacgc tgaacggcga agccgtggcc | 180 |
| gccatcgcac cgatgccccc gggtgcaccg gaggggatgc cgccgatctg gaacacctat | 240 |
| atcgcggtgg acgacgtcga tgcggtggtg gacaaggtgg tgcccggggg cgggcaggtg | 300 |
| atgatgccgg ccttcgacat cggcgatgcc ggccggatgt cgttcatcac cgatccgacc | 360 |
| ggcgctgccg tgggcctatg gcaggccaat cggcacatcg gagcgacgtt ggtcaacgag | 420 |
| acgggcacgc tcatctggaa cgaactgctc acggacaagc cggatttggc gctagcgttc | 480 |
| tacgaggctg tggttggcct cacccactcg agcatggaga tagctgcggg ccagaactat | 540 |
| cgggtgctca aggccggcga cgcggaagtc ggcggctgta tggaaccgcc gatgcccggc | 600 |
| gtgccgaatc attggcacgt ctactttgcg gtggatgacg ccgacgccac ggcggccaaa | 660 |
| gccgccgcag cgggcggcca ggtcattgcg gaaccggctg acattccgtc ggtgggccgg | 720 |
| ttcgccgtgt tgtccgatcc gcagggcgcg atcttcagtg tgttgaagcc cgcaccgcag | 780 |
| caatag | 786 |

This exemplary nucleic acid molecule, Rv0577 herein, is a tubercle-complex specific gene that was cloned and isolated from Mycobacterium tuberculosis (Mtb) H37Rv. Rv0577 has been identified as a 786 nucleotide cDNA, encoding a protein of 261 amino acids (plus the stop codon). Also suitable in the nucleic acid construct of the present invention is a nucleic acid molecule having a nucleotide sequence that hybridizes to the nucleic acid molecule corresponding to SEQ ID NO: 1 under stringent conditions. For the purposes of defining the level of stringency, reference can conveniently be made to Sambrook et al., Molecular Cloning: A Laboratory Manual, Third Edition, Cold Spring Harbor: Cold Spring Harbor Laboratory Press, New York (2001)(which is hereby incorporated by reference in its entirety). An example of high stringency conditions includes 4-5×SSC/0.1% w/v SDS at 54° C. for 1-3 hours. Another stringent hybridization condition is hybridization at 4×SSC at 65° C., followed by a washing in 0.1×SSC at 65° C. for about one hour. Alternatively, an exemplary stringent hybridization condition is in 50% formamide, 4×SSC, at 42° C. Still another example of stringent conditions include hybridization at 62° C. in 6×SSC, 0.05× BLOTTO, and washing at 2×SSC, 0.1% SDS at 62° C. The skilled artisan is aware of various parameters which may be altered during hybridization and washing and which will either maintain or change the stringency conditions.

Also suitable in the nucleic acid construct of the present invention is a nucleic acid molecule having a nucleotide sequence wherein the nucleic acid molecule is at least 55% similar to the nucleotide sequence of SEQ ID NO: 1 by basic BLAST using default parameters analysis (Altschul et al., “Gapped BLAST and PSI-BLAST: a New Generation of Protein Database Search Programs,” Nucleic Acids Res. 25:3389-3402 (1997), which is hereby incorporated by reference in its entirety).

In one aspect of the present invention, the nucleic acid construct of the present invention has an nucleic acid molecule that encodes a protein or polypeptide having an amino acid sequence corresponding to SEQ ID NO: 2, as follows:

```
Met Pro Lys Arg Ser Glu Tyr Arg Gln Gly Thr Pro Asn Trp Val Asp
    1 5 10 15
Leu Gln Thr Thr Asp Gln Ser Ala Ala Lys Lys Phe Tyr Thr Ser Leu
                          20 25 30
Phe Gly Trp Gly Tyr Asp Asp Asn Pro Val Pro Gly Gly Gly Gly Val
                  35 40 45
Tyr Ser Met Ala Thr Leu Asn Gly Glu Ala Val Ala Ala Ile Ala Pro
          50 55 60
Met Pro Pro Gly Ala Pro Glu Gly Met Pro Pro Ile Trp Asn Thr Tyr
  65 70 75 80
Ile Ala Val Asp Asp Val Asp Ala Val Val Asp Lys Val Val Pro Gly
                                  85 90 95
Gly Gly Gln Val Met Met Pro Ala Phe Asp Ile Gly Asp Ala Gly Arg
                        100 105 110
Met Ser Phe Ile Thr Asp Pro Thr Gly Ala Ala Val Gly Leu Trp Gln
                115 120 125
Ala Asn Arg His Ile Gly Ala Thr Leu Val Asn Glu Thr Gly Thr Leu
        130 135 140
Ile Trp Asn Glu Leu Leu Thr Asp Lys Pro Asp Leu Ala Leu Ala Phe
145 150 155 160
Tyr Glu Ala Val Val Gly Leu Thr His Ser Ser Met Glu Ile Ala Ala
                                165 170 175
Gly Gln Asn Tyr Arg Val Leu Lys Ala Gly Asp Ala Glu Val Gly Gly
                        180 185 190
Cys Met Glu Pro Pro Met Pro Gly Val Pro Asn His Trp His Val Tyr
                195 200 205
Phe Ala Val Asp Asp Ala Asp Ala Thr Ala Ala Lys Ala Ala Ala Ala
        210 215 220
Gly Gly Gln Val Ile Ala Glu Pro Ala Asp Ile Pro Ser Val Gly Arg
225 230 235 240
Phe Ala Val Leu Ser Asp Pro Gln Gly Ala Ile Phe Ser Val Leu Lys
                                245 250 255
Pro Ala Pro Gln Gln
                        260
```

The Rv0577 protein or polypeptide, termed Rv0577 or CFP32 herein, is the ˜32 kDa, IL-10 producing protein encoded by Rv0577.

The protein or polypeptide of the present invention is preferably produced in purified form by conventional techniques. Typically, the protein or polypeptide of the present invention is secreted into the growth medium of recombinant E. coli. To isolate the protein, the E. coli host cell carrying a recombinant plasmid is propagated, homogenized, and the homogenate is centrifuged to remove bacterial debris. The supernatant is then subjected to sequential ammonium sulfate precipitation. The fraction containing the protein of the present invention is subjected to gel filtration in an appropriately sized dextran or polyacrylamide column to separate the proteins. If necessary, the protein fraction may be further purified by HPLC. Alternative methods may be used as suitable. Mutations or variants of the above polypeptide or protein are encompassed by the present invention.

Variants may be modified by, for example, the deletion or addition of amino acids that have minimal influence on the properties, secondary structure, and hydropathic nature of the polypeptide. For example, a polypeptide may be conjugated to a signal (or leader) sequence at the N-terminal end of the protein which co-translationally or post-translationally directs transfer of the protein. The polypeptide may also be conjugated to a linker or other sequence for ease of synthesis, purification, or identification of the polypeptide.

Fragments of the above protein are also encompassed by the present invention. Suitable fragments can be produced by several means. In the first, subclones of the gene encoding the protein of the present invention are produced by conventional molecular genetic manipulation by subcloning gene fragments. The subelones then are expressed in vitro or in vivo in bacterial cells to yield a smaller protein or peptide.

In another approach, based on knowledge of the primary structure of the protein of the present invention, fragments of the gene of the present invention may be synthesized by using the PCR technique together with specific sets of primers chosen to represent particular portions of the protein. These then would be cloned into an appropriate vector for increased expression of an accessory peptide or protein.

Chemical synthesis can also be used to make suitable fragments. Such a synthesis is carried out using known amino acid sequence for the protein of the present invention. These fragments can then be separated by conventional procedures (e.g., chromatography, SDS-PAGE) and used in the methods of the present invention.

The making of a nucleic acid construct of the present invention generally involves first inserting the desired nucleic acid molecule into an expression system to which the nucleic acid molecule is heterologous (ie., not normally present). The heterologous nucleic acid molecule is inserted into the expression system which includes the necessary elements for the transcription and translation of the inserted protein coding sequences.

The nucleic acid molecule(s) of the present invention may be inserted into any of the many available expression vectors using reagents that are well known in the art. In preparing the nucleic acid constructs of the present invention, the various nucleic acid molecules of the present invention may be inserted or substituted into a bacterial plasmid-vector. Any convenient plasmid may be employed, which will be characterized by having a bacterial replication system, a marker which allows for selection in a bacterium and generally one or more unique, conveniently located restriction sites. Numerous plasmids, referred to as transformation vectors, are available for transformation. Suitable vectors include, but are not limited to, the following: viral vectors, such as lambda vector system gt11, gt WES.tB, Charon 4, and plasmid vectors such as pBR322, pBR325, pACYC177, pACYC1084, pUC8, pUC9, pUC18, pUC19, pLG339, pR290, pKC37, pKC101, SV 40, pBluescript II SK ± or KS ± (see “Stratagene Cloning Systems” Catalog (1993) from Stratagene, La Jolla, Calif., which is hereby incorporated by reference in its entirety), pQE, pIH821, pGEX, pET series (see F. W. Studier et. al., “Use of T7 RNA Polymerase to Direct Expression of Cloned Genes,” Gene Expression Technology vol. 185 (1990), which is hereby incorporated by reference in its entirety), and any derivatives thereof. The selection of a vector will depend on the preferred transformation technique and target cells for transfection.

Certain “control elements” or “regulatory sequences” are also incorporated into the plasmid-vector constructs of the present invention. These include non-transcribed regions of the vector and 5′ and 3′ untranslated regions, which interact with host cellular proteins to carry out transcription and translation. Such elements may vary in their strength and specificity. Depending on the vector system and host utilized, any number of suitable transcription and/or translation elements, including constitutive, inducible, and repressible promoters, as well as minimal 5′ promoter elements may be used.

A constitutive promoter is a promoter that directs constant expression of a gene in a cell. Examples of some constitutive promoters that are widely used for inducing expression of transgenes include the nopoline synthase (“NOS”) gene promoter, from Agrobacterium tumefaciens (U.S. Pat. No. 5,034,322 issued to Rogers et al., which is hereby incorporated by reference in its entirety), the cauliflower mosaic virus (“CaMV”) 35S and 19S promoters (U.S. Pat. No. 5,352,605 issued to Fraley et al., which is hereby incorporated by reference in its entirety), those derived from any of the several actin genes, which are known to be expressed in most cells types (U.S. Pat. No. 6,002,068 issued to Privalle et al., which is hereby incorporated by reference in its entirety), and the ubiquitin promoter (“ubi”), which is the promoter of a gene product known to accumulate in many cell types. Examples of constitutive promoters for use in mammalian cells include the RSV promoter derived from Rous sarcoma virus, the CMV promoter derived from cytomegalovirus, β-actin and other actin promoters, and the EF1α promoter derived from the cellular elongation factor 1α gene.

Also suitable as a promoter in the plasmids of the present invention is a promoter that allows for external control over the regulation of gene expression. One way to regulate the amount and the timing of gene expression is to use an inducible promoter. Unlike a constitutive promoter, an inducible promoter is not always optimally active. An inducible promoter is capable of directly or indirectly activating transcription of one or more DNA sequences or genes in response to an inducer. Some inducible promoters are activated by physical means such as the heat shock promoter (“Hsp”). Others are activated by a chemical, for example, IPTG or tetracycline (“Tet on” system). Other examples of inducible promoters include the metallothionine promoter, which is activated by heavy metal ions, and hormone-responsive promoters, which are activated by treatment of certain hormones. In the absence of an inducer, the nucleic acid sequences or genes under the control of the inducible promoter will not be transcribed or will only be minimally transcribed. When any plasmids of the present invention contain an inducible promoter, the method of the present invention further includes the step of adding an appropriate inducing agent to the cell culture when activation of the promoter is desired. Promoters of the nucleic acid construct of the present invention may be either homologous (derived from the same species as the host cell) or heterologous (derived from a different species than the host cell).

The nucleic acid molecule of the present invention, a promoter molecule of choice, a suitable 3′ regulatory region, and if desired, a reporter gene, are incorporated into a vector-expression system of choice to prepare the nucleic acid construct of present invention using standard cloning procedures known in the art, such as described by Sambrook et al., Molecular Cloning: A Laboratory Manual, Third Edition, Cold Spring Harbor: Cold Spring Harbor Laboratory Press, New York (2001), which is hereby incorporated by reference in its entirety, and U.S. Pat. No. 4,237,224 to Cohen and Boyer, which is hereby incorporated by reference in its entirety, which describes the production of expression systems in the form of recombinant plasmids using restriction enzyme cleavage and ligation with DNA ligase. These recombinant plasmids are then introduced by means of transformation and replicated in unicellular cultures including prokaryotic organisms and eukaryotic cells grown in tissue culture.

In one aspect of the present invention, a nucleic acid molecule encoding a protein of choice is inserted into a vector in the sense (i.e., 5′→3′) direction, such that the open reading frame is properly oriented for the expression of the encoded protein under the control of a promoter of choice. Single or multiple nucleic acids may be ligated into an appropriate vector in this way, under the control of a suitable promoters, to prepare a nucleic acid construct of the present invention. In another aspect, the nucleic acid molecule is inserted into the expression system or vector in the antisense (i.e., 3′→5′) orientation. The antisense form of the nucleic acid molecule is complementary to the Rv0577 nucleic acid molecule of the present invention, or complementary to a fragment of the Rv0577 nucleic acid molecule.

Once the nucleic acid construct of the present invention has been prepared, it is ready to be incorporated into a host cell. Accordingly, another aspect of the present invention relates to a recombinant cell, or “host” cell containing the nucleic acid construct of the present invention. Basically, this is carried out by transforming or transfecting a host cell with a plasmid construct of the present invention, using standard procedures known in the art, such as described by Sambrook et al., Molecular Cloning: A Laboratory Manual, Third Edition, Cold Spring Harbor: Cold Spring Harbor Laboratory Press, New York (2001), which is hereby incorporated by reference in its entirety. Suitable host cells for the present invention include, without limitation, bacterial cells, virus, yeast cells, plant cells, and mammalian cells, including human cells, as well as any other cell system that is suitable for producing a recombinant protein. Methods of transformation or transfection may result in transient or stable expression of the genes of interest contained in the plasmids. After transformation, the transformed host cells can be selected and expanded in suitable culture. Preferably, transformed cells are first identified using a selection marker simultaneously introduced into the host cells along with the nucleic acid construct of the present invention. Suitable markers include markers encoding for antibiotic resistance, such as the nptII gene which confers kanamycin resistance (Fraley, et al., Proc. Natl. Acad. Sci. USA, 80:4803-4807 (1983), which is hereby incorporated by reference in its entirety), or gentamycin, G418, ampicillin, hygromycin, streptomycin, spectinomycin, tetracycline, chloramphenicol, and the like (Southern and Berg, “Transformation of Mammalian Cells to Antibiotic Resistance With a Bacterial Gene Under the Control of the SV40 Early Region Promoter,” J Mol Appl Genet., 1(4):327-41 (1982); Bernard et al., “Construction of a Fusion Gene That Confers Resistance Against Hygromycin B to Mammalian Cells in Culture,” Exp Cell Res. 158(1):237-43 (1985), which are hereby incorporated by reference in their entirety). A number of antibiotic-resistance markers are known in the art and others are continually being identified. Any known antibiotic-resistance marker can be used to transform and select transformed host cells in accordance with the present invention. Cells or tissues are grown on a selection medium containing an antibiotic, whereby generally only those transformants expressing the antibiotic resistance marker continue to grow. Additionally, or in the alternative, reporter genes, including, but not limited to, β-Glucuronidase, luciferase, green fluorescent protein (GFP) or enhanced green fluorescent protein (EGFP), may be used for selection of transformed cells. The selection marker employed will depend on the target species; for certain target species, different antibiotics, or biosynthesis selection markers are preferable.

The present invention also relates to an isolated antibody, or binding portion thereof, against a protein or polypeptide having an amino acid sequence corresponding to SEQ ID NO: 2 of the present invention. This aspect of the present invention involves producing antibodies against the polypeptide or protein of the present invention that are capable of inhibiting the activity of a polypeptide or protein of the present invention. The antibodies of the present invention may be monoclonal or polyclonal. Monoclonal antibody production may be effected by techniques which are well-known in the art. Basically, the process involves first obtaining immune cells (lymphocytes) from the spleen of a mammal (e.g., mouse) which has been previously immunized with the antigen of interest either in vivo or in vitro. The antibody-secreting lymphocytes are then fused with (mouse) myeloma cells or transformed cells, which are capable of replicating indefinitely in cell culture, thereby producing an immortal, immunoglobulin-secreting cell line. The resulting fused cells, or hybridomas, are cultured, and the resulting colonies screened for the production of the desired monoclonal antibodies. Colonies producing such antibodies are cloned, and grown either in vivo or in vitro to produce large quantities of antibody. A description of the theoretical basis and practical methodology of fusing such cells is set forth in Kohler and Milstein, “Continuous Culture of Fused Cells Secreting Antibody of Predefined Specificity,” Nature, 256:495-7 (1975), which is hereby incorporated by reference in its entirety.

Mammalian lymphocytes are immunized by in vivo immunization of the animal (e.g., a mouse) with the protein or polypeptide of the present invention. Such immunizations are repeated as necessary at intervals of up to several weeks to obtain a sufficient titer of antibodies. Following the last antigen boost, the animals are sacrificed and spleen cells removed.

Fusion with mammalian myeloma cells or other fusion partners capable of replicating indefinitely in cell culture is effected by standard and well-known techniques, for example, by using polyethylene glycol (“PEG”) or other fusing agents (Milstein et al., “Derivation of Specific Antibody-Producing Tissue Culture and Tumor Lines by Cell Fusion,” Eur. J. Immunol., 6:511-19 (1976), which is hereby incorporated by reference in its entirety). This immortal cell line, which may be derived from cells of any mammalian species, including, but not limited to, mouse, rat, and human, is selected to be deficient in enzymes necessary for the utilization of certain nutrients, to be capable of rapid growth, and to have good fusion capability. Many such cell lines are known to those skilled in the art, and others are regularly described.

Procedures for raising polyclonal antibodies are also well known. Typically, such antibodies can be raised by administering the protein or polypeptide of the present invention subcutaneously to New Zealand white rabbits which have first been bled to obtain pre-immune serum. The antigens can be injected at a total volume of 100 μl per site at six different sites. Each injected material will contain synthetic surfactant adjuvant pluronic polyols, or pulverized acrylamide gel containing the protein or polypeptide after SDS-polyacrylamide gel electrophoresis. The rabbits are then bled two weeks after the first injection and periodically boosted with the same antigen three times every six weeks. A sample of serum is then collected 10 days after each boost. Polyclonal antibodies are then recovered from the serum by affinity chromatography using the corresponding antigen to capture the antibody. Ultimately, the rabbits are euthenized with pentobarbital 150 mg/Kg IV. This and other procedures for raising polyclonal antibodies are disclosed in E. Harlow, et. al., Editors, Antibodies: a Laboratory Manual (1988), which is hereby incorporated by reference in its entirety.

Another aspect of the present invention is a method for detection of Mycobacterium tuberculosis specific antibodies in a sample of tissue or body fluids. This method involves providing the isolated protein or polypeptide of the present invention as an antigen; contacting the sample with the antigen; contacting the sample with the antigen under conditions effective to allow formation of a complex of the antigen bound to antibodies which recognize the antigen; and detecting if any of the complex is present, thereby indicating a presence of Mycobacterium tuberculosis the sample. Body fluids suitable for this aspect of the present invention include blood, saliva, sputum, and pulmonary lavage fluid. In this aspect of the present invention, the protein or polypeptide may have a label to permit detection of binding of the antibody in a biological sample, including a tissue or body fluid. Suitable labels include a fluorescent label, a radioactive label, a nuclear magnetic resonance active label, a luminescent label, and a chromophore label. Any assay system capable of detecting a complex of the antigen bound to antibodies which recognize the antigen is suitable for this aspect of the present invention, including, but not limited to, an enzyme-linked immunosorbent assay, a radioimmunoassay, a gel diffusion precipitin reaction assay, an immunodiffusion assay, an agglutination assay, a fluorescent immunoassay, a protein A imnmunoassay, and an immunoelectrophoresis assay.

The present invention also relates to another method for detection of Mycobacterium tuberculosis in a sample of tissue or body fluids. This method involves providing an antibody or binding portion thereof against the protein or polypeptide of the present invention, contacting the sample with the antibody or binding portion thereof under conditions effective to allow formation of a complex of the antibody or binding portion thereof and an antigen recognized by the antibody or binding portion thereof, and detecting if any of the complex is present, thereby indicating the presence of Mycobacterium tuberculosis in the sample. As indicated above, antibodies suitable for use in accordance with the present invention include monoclonal or polyclonal antibodies. In addition, antibody fragments, half-antibodies, hybrid derivatives, probes, and other molecular constructs may be utilized. Also suitable in this aspect of the present invention are binding portions of such antibodies. Such binding portions include Fab fragments, F(ab′)₂ fragments, and Fv fragments. These antibody fragments can be made by conventional procedures, such as proteolytic fragmentation procedures, as described in J. Goding, Monoclonal Antibodies: Principles and Practice, pp. 98-118 (N.Y. Academic Press 1983), which is hereby incorporated by reference in its entirety. Detecting may be carried out by any assay system capable of detecting a complex of the antibody or binding portion thereof and an antigen recognized by the antibody or binding portion, including, but not limited to, those described supra. The antibody or binding portion thereof may be labeled as describe supra, for use in a suitable assay system.

The present invention also relates to a third method for detection of Mycobacterium tuberculosis in a sample of tissue or body fluids. This method involves providing a nucleic acid molecule of the present invention as a probe in a nucleic acid hybridization assay; contacting the sample with the probe under conditions effective to permit formation of a complex of the probe and nucleic acid which hybridizes to the probe; and detecting formation of the complex in the sample thereby indicating a presence of Mycobacterium tuberculosis in the sample. Methods of detection may include, but are not limited to, electrophoresis, DNA sequencing, blotting, and in-situ hybridization.

The present invention also relates to a fourth method of detection of Mycobacterium tuberculosis in a sample of tissue or body fluids. This method involves providing a nucleic acid molecule of the present invention as a probe or primer in a gene amplification detection procedure, contacting the sample with the probe or primer under conditions effective to amplify probe or primer-specific nucleic acid molecules, and detecting any amplified probe or primer-specific molecules, thereby indicating a presence of Mycobacterium tuberculosis in the sample. A number of methods can be used to amplify the nucleic acid molecule encoding the protein of the present invention. These include, but are not limited to, polymerase chain reaction (PCR), ligase chain reaction (LCR), ligase detection reaction (LDR), LDR-PCR, strand displacement amplification, hybridization signal amplification (HSAM), self-sustained sequence (3SR) replication, Q-beta replicase, nucleic acid sequence based amplification (“NASBA”), transcription-based amplification System (“TAS”), or branched-DNA methods. Detection methods include any methods commonly associated with the method of amplification carried out, including, but not limited to, gel electrophoresis, array-capture, and direct sequencing. The nucleic acid probes in this aspect of the present invention can be labeled or tagged in accordance with the detection method of choice.

The present invention also relates to a method of vaccinating a mammal against infection by Mycobacterium tuberculosis. This method involves administering an effective amount of the isolated protein or polypeptide having an amino acid sequence corresponding to SEQ ID NO: 2 of the present invention to the mammal. In this and all other aspects of the present invention “administering” may be oral, intradermal, intramuscular, intraperitoneal, intravenous, subcutaneous, or intranasal. Oral immunization offers certain advantages over other routes of vaccination. For example, oral vaccines are more easily administered and, therefore, may be more acceptable to vaccine recipients. Also, oral vaccines can be less pure than vaccines formulated for injection, making production costs lower. Oral vaccines may also include flavorings, colorings, and other food additives to make the vaccine more palatable. In addition, oral vaccines may also contain stabilizers and preservatives to extend the shelf life of the vaccine.

The proteins or polypeptides which are to be administered as vaccines according to the present invention can be formulated according to conventional and/or future methods for such administration to the subject to be protected and can be mixed with conventional adjuvants. The peptide expressed can be used as an immunogen in subunit vaccine formulations, which may be multivalent. The product may be purified for purposes of vaccine formulation from any vector/host systems that express the heterologous protein. The purified protein or polypeptide of the present invention should be adjusted to an appropriate concentration, formulated with any suitable vaccine adjuvant and packaged for use. Suitable adjuvants include, but are not limited to: mineral gels, e.g., aluminum hydroxide; surface active substances such as lysolecithin, pluronic polyols; polyanions; peptides; oil emulsions; and potentially useful human adjuvants such as BCG (Bacille Calmette-Guerin) and Corynebacterium parvum. The immunogen may also be incorporated into liposomes, or conjugated to polysaccharides and/or other polymers for use in a vaccine formulation.

Another aspect of the present invention is a vaccine for preventing infection and disease of mammals by Mycobacterium tuberculosis. This vaccine includes an isolated protein or polypeptide having an amino acid sequence corresponding to SEQ ID NO: 2 of the present invention, and a pharmaceutically-acceptable carrier.

The present invention also relates to another method of vaccinating mammals against infection by Mycobacterium tuberculosis. This involves administering an effective amount of the vaccine according to the present invention to mammals.

The present invention also relates to a method of treating mammals infected with Mycobacterium tuberculosis. This method involves administering an effective amount of the antibody, or a binding portion thereof, against the protein or polypeptide of the present invention to mammals infected with Mycobacterium tuberculosis. A suitable antibody of this aspect of the present invention may be a monoclonal or polyclonal antibody, or a binding portion thereof, all as described above herein. Administering of such an antibody or a binding portion thereof may be oral, intradermal, intramuscular, intraperitoneal, intravenous, subcutaneous, or intranasal. The antibodies or binding portions thereof in accordance with this and all other aspects of the present invention in which antibodies are administered to a mammal for prevention or treatment of Mycobacterium tuberculosis infection can be administered orally, parenterally, subcutaneously, intravenously, intramuscularly, intraperitoneally, by intranasal instillation, by intracavitary or intravesical instillation, intraocularly, intraarterially, intralesionally, or by application to mucous membranes, such as, that of the nose, throat, and bronchial tubes. They may be administered alone or with pharmaceutically or physiologically acceptable carriers, excipients, or stabilizers, and can be in solid or liquid form such as, tablets, capsules, powders, solutions, suspensions, or emulsions.

The solid unit dosage forms can be of the conventional type. The solid form can be a capsule, such as an ordinary gelatin type containing the antibodies or binding portions thereof of the present invention and a carrier, for example, lubricants and inert fillers such as, lactose, sucrose, or cornstarch. In another embodiment, these compounds are tableted with conventional tablet bases such as lactose, sucrose, or cornstarch in combination with binders like acacia, cornstarch, or gelatin, disintegrating agents, such as cornstarch, potato starch, or alginic acid, and a lubricant, like stearic acid or magnesium stearate.

The antibody or binding portion thereof of the present invention may also be administered in injectable dosages by solution or suspension of these materials in a physiologically acceptable diluent with a pharmaceutical carrier. Such carriers include sterile liquids, such as water and oils, with or without the addition of a surfactant and other pharmaceutically and physiologically acceptable carrier, including adjuvants, excipients or stabilizers. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions.

For use as aerosols, the antibody or binding portion thereof of the present invention in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

The present invention also relates to a composition for passively immunizing mammals infected with Mycobacterium tuberculosis. This composition includes an isolated antibody, or binding portion thereof, according the present invention and a pharmaceutically-acceptable carrier. A suitable antibody of this aspect of the present invention may be a monoclonal or polyclonal antibody or a binding portion thereof, prepared as described herein, and where the administration of the composition is as described herein, supra.

The present invention also relates a method of passively immunizing mammals infected with Mycobacterium tuberculosis. This method involves administering an effective amount of the composition of the present invention having the isolated antibody, or binding portion thereof, according the present invention and a pharmaceutically-acceptable carrier to mammals infected with Mycobacterium tuberculosis. Suitable antibodies and the administration thereof are as described herein, supra.

Another aspect of the present invention relates to a method of enhancing vaccination against Mycobacterium tuberculosis using a composition comprising a microorganism capable of producing an antigenic response against Mycobacterium tuberculosis when introduced into a host subject. This method involves suppressing in the microorganism the expression of a nucleic acid molecule that either: 1) has a nucleotide sequence corresponding to SEQ ID NO: 1; 2) has a nucleotide sequence that hybridizes to the nucleic acid corresponding to SEQ ID NO: 1 under stringent conditions characterized by a hybridization buffer comprising 5×SSC at a temperature of 54° C.; 3) is at least 55% similar to the nucleotide sequence of SEQ ID NO: 1 by basic BLAST using default parameters analysis; or 4) encodes a protein or polypeptide having an amino acid sequence corresponding to SEQ ID NO: 2. A microorganism suitable for use in this aspect is Mycobacterium bovis Bacillus Calmette-Guerin.

As described in greater detail in the Examples below, the protein encoded by Rv0577 (RV0577/CFP32) is capable of suppressing the immune response in subjects infected with Mtb. Therefore, it is advantageous to interfere with the production of RV0577/CFP32 in a subject, thereby diminishing the dampening of immune response in an Mtb-infected subject. This can be carried out in a variety of ways. For example, a genetically modified microorganism for this aspect of the present invention can be prepared using the knowledge provided herein with regard to the Rv0577 gene and its protein product, in combination with conventional molecular biology techniques for manipulation of the nucleotide sequence thereof, and insertion into an appropriate expression vector for use in this aspect of the present invention (e.g., Sambrook et al., Molecular Cloning: A Laboratory Manual, Third Edition, Cold Spring Harbor: Cold Spring Harbor Laboratory Press, New York (2001); Parish et al., “glnE Is An Essential Gene in Mycobacterium tuberculosis,” J. Bacteriol. 182(20): 5715-5720 (2000), which are hereby incorporated by reference in their entirety), in accordance with common conventions for production of a composition for use in active immunization (e.g., Kuby, J., Immunology, W. H. Freeman Co., New York, N.Y. Chap. 18 (1992), which is hereby incorporated by reference in its entirety). Regulation of the expression of the nucleic acid molecule of the present invention involves transformation of a cell or tissue of choice, either in vivo or ex vivo with a suitable nucleic acid construct of the present invention. In this aspect of the present invention, in which suppression (including ablation) of expression of Cfp32 is desired, this method involves preparing a recombinant mycobacterium having the Cfp32-encoding nucleotide sequence removed from the microorganism. Alternatively, the nucleic acid construct of the present invention may be configured so that the nucleic acid molecule encodes an mRNA which is not translatable, i.e., does not result in the production of a protein or polypeptide. This is achieved, for example, by introducing into the desired nucleic acid sequence of the present invention one or more premature stop codons, adding one or more bases (except multiples of 3 bases) to displace the reading frame, and removing the translation initiation codon (U.S. Pat. No. 5,583,021 to Dougherty et al., which is hereby incorporated by reference in its entirety). This can involve the use of a primer to which a stop codon, such as TAATGA, is inserted into the sense (or “forward”) PCR-primer for amplification of the full nucleic acid, between the 5′ end of that primer, which corresponds to the appropriate restriction enzyme site of the vector into which the nucleic acid is to be inserted, and the 3′ end of the primer, which corresponds to the 5′ sequence of the enzyme-encoding nucleic acid.

Genes can be effective in the non-translatable antisense forms, as well as in the non-translatable sense form (Baulcombe, D. C., “Mechanisms of Pathogen-Derived Resistance to Viruses in Transgenic Plants,” Plant Cell 8:1833-44 (1996); Dougherty, W. G., et al., “Transgenes and Gene Suppression: Telling us Something New?” Current Opinion in Cell Biology 7:399-05 (1995); Lomonossoff, G. P., “Pathogen-Derived Resistance to Plant Viruses,” Ann. Rev. Phytopathol. 33:323-43 (1995), which are hereby incorporated by reference in their entirety).

Alternatively, a suitable construct for this aspect of the present invention includes the nucleic acid molecule of the present invention placed in a suitable vector in an antisense orientation, as described above. The use of antisense RNA to down-regulate the expression of specific genes is well known (van der Krol et al., Nature, 333:866-869 (1988) and Smith et al., Nature 334:724-726 (1988), which are hereby incorporated by reference in their entirety). Antisense nucleic acids are DNA or RNA molecules that are complementary to at least a portion of a specific mRNA molecule (Weintraub, “Antisense RNA and DNA,” Scientific American 262:40 (1990), which is hereby incorporated by reference in its entirety). Antisense methodology takes advantage of the fact that nucleic acids tend to pair with “complementary” sequences. By complementary, it is meant that polynucleotides are capable of base-pairing according to the standard Watson-Crick rules. In the target cell, the antisense nucleic acids hybridize to a target nucleic acid and interferes with transcription, and/or RNA processing, transport, translation, and/or stability. The overall effect of such interference with the target nucleic acid function is the disruption of protein expression.

The present invention also relates to another composition for actively immunizing mammals against Mycobacterium tuberculosis. This composition has a microorganism capable of producing an antigenic response against Mycobacterium tuberculosis, where the microorganism has been modified to be incapable of producing a nucleic acid molecule encoding a factor suppressing an immune response to Mycobacterium tuberculosis in a host, and a pharmaceutically-acceptable carrier. This composition involves removing or turning off the Rv0577 gene in an organism (i.e., making the gene incapable of normal transcription and/or translation) as described herein above, or as described in the art, which organism is then used for producing an antigenic response to Mtb in a mammalian. An exemplary microorganism for this aspect of the present invention is M. bovis BCG, the attenuated strain of tubercle bacillus widely used in the vaccination of humans against tuberculosis. The modification to the microorganism involves the application of standard molecular biology procedures known in the art, for example, the making of a null mutant microorganism, such as describe in Example 3 here, or those described by Sambrook et al., Molecular Cloning: A Laboratory Manual, Third Edition, Cold Spring Harbor: Cold Spring Harbor Laboratory Press, New York (2001)(which is hereby incorporated by reference in its entirety).

As described in the Background, supra, Il -10 is a potent inhibitor of inflammatory response in mammals. Thus, another aspect of the present invention is a method of treating inflammatory disease in a mammal. This method involves administering a nucleic acid construct of the present invention having a nucleic acid molecule that encodes a factor suppressing an immune response to Mycobacterium tuberculosis such that the nucleic acid molecule, when expressed in the mammal, suppresses an inflammatory response; and operably linked 5′ and 3′ regulatory elements; and administering the nucleic acid construct to a mammal under conditions effective to treat an inflammatory disease. Preferably, the nucleic acid molecule of this aspect of the present invention is a nucleic acid molecule that either: (1) has a nucleotide sequence corresponding to SEQ ID NO: 1 of the present invention, (2) has a nucleotide sequence that hybridizes to the nucleic acid corresponding to SEQ ID NO: 1 under stringent conditions characterized by a hybridization buffer comprising 5×SSC at a temperature of 54° C., (3) is at least 55% similar to the nucleotide sequence of SEQ ID NO: 1 by basic BLAST using default parameters analysis, or (4) encodes a protein or polypeptide having an amino acid sequence of SEQ ID NO: 2 of the present invention. “Administering” of the nucleic acid molecule in this aspect of the present invention may be oral, intradermal, intramuscular, intraperitoneal, intravenous, subcutaneous, or intranasal. This aspect of the present invention involves the treatment of inflammatory diseases including, but not limited to, bronchiectasis, asthma (Nahori et al., “Effects of Mycobacterium bovis BCG on the Development of Allergic Inflammation and Bronchial Hyperresponsiveness in Hyper-IgE BP2 Mice Vaccinated as Newborns,” Vaccine 19:1484-1495 (2001), which is hereby incorporated by reference in its entirety) and sepsis; autoimmune diseases such as lupus, rheumatoid arthritis, and scleroderma; inflammatory bowel diseases; multiple sclerosis; and tropical spastic paralysis.

The present invention also relates to another method of treating inflammatory disease in a mammal. This method involves providing a protein or polypeptide that suppresses an immune response to Mycobacterium tuberculosis; and administering the protein or polypeptide to a mammal under conditions effective to treat inflammatory disease. The preferable protein or polypeptide of this aspect of the present invention has an amino acid sequence corresponding to SEQ ID NO: 2 of the present invention. Diseases that may be treated by this method include, but are not limited to, those described in the preceding paragraph.

### EXAMPLES

### Example 1

Culture of Mycobacteria and Source of Mycobacteria Components

M. smegmatis (Ms; ATCC 23038 (lots 961 and 972) or mc²155), Mtb H37Rv (MtbRv, ATCC 27294 (lots 013, 082)), MtbH37Ra (MtbRa, ATCC 25177 (lot 082)), M. bovis Bacillus Calmette-Guerin (BCG, ATCC 27290, vaccine strain Copenhagen) were grown in Middlebrook 7H9 broth (Difco Laboratory, Detroit, Mich.) supplemented with 0.2% glycerol, 0.5% Tween 80, and 10% Middlebrook ADC (Difco Laboratory). Hygromycin B (50 μg/ml) was added for the selection of 577 null mutant or M. smegmatis transformed with pMS3 or pMS3.577 and kanamycin (25 μg/ml) was added for the selection of 577 null Mtb complemented with pMSG.577 or M. smegmatis transformed with pMSG.577. Agar cultures were grown in 7H11 medium supplemented with 0.2% glycerol, 0.5% Tween 80, and 10% Middlebrook OADC (and in some cultures with 100 μg/ml cycloheximide). Growth over 23 days in liquid medium ofthe parental, 577 null mutant, and complemented 577 null mutant Mtb were quantitated by absorbance at 580 nm, and by plating of serial dilutions of the broth culture onto 7H 11 agar as indicated above. The initial cultures were started using frozen glycerol stocks by inoculating with approximately 2×10⁵ CFU/ml in a 30 ml supplemented Middlebrook 7H9 broth as indicated above. Mtb H37Rv components (whole cell lysate, membrane, cytosol, cell wall, culture filtrate (CFP), and CFP fractions obtained by anion exchange (QAE chromatography) and mannose capped lipoarabinomannan (manLam) were obtained from Dr. John Belisle (NIH Mtb Reagents Program, Colorado State University, Fort Collins, Colo.). Purified protein derivative (PPD) without preservatives was obtained from Adventis Pasteur (formerly Pasteur, Merieux, Connaught), Swiftwater, Pa. All Mtb reagents shown were tested for endotoxin (lipopolysaccharide, LPS) by the Limulus amebocyte assay (BioWhittiker, Walkersville, Md.) and contained less than 50 pg of LPS per μg of reagent or per 106 CFU of mycobacteria.

### Example 2

Human Monocyte Isolation, Culture, and Challenge with Mycobacteria Components

Human monocytes were purified from peripheral blood mononuclear cells (PBMC) obtained as leukocyte enriched packs (New York Blood Center, New York, N.Y.). Purification was performed by either self-agglutination ( Mentzer et al., “Spontaneous Aggregation as a Mechanism for Human Monocyte Purification,” Cell. Immunol. 10(2):312-19 (1986), which is hereby incorporated by reference in its entirety) and cultured in RPMI 1640 medium supplemented with 100 g/ml streptomycin, 100 U/ml penicillin and 100 g/ml L-glutamine, or by negative selection (immunodepletion) using manufacturer's protocol (StemCell Technologies, Vancouver, Canada) and cultured in X-Vivo-20 medium (Bio-Whittiker, Wakerfield, Md., an artificial medium without antibiotics, serum nor LPS). Human monocytes were stimulated with Ms, BCG, Mtb, or Mtb components on day of isolation or after 24 h in culture. Cell free supernatants obtained 48 h after challenge with mycobacteria or Mtb components and stored in −80 ° C. were assayed for cytokines (IL-10, TNF-α, or IL-1β) using commercial kits or antibody pairs as directed by the manufacturer

### Example 3

Construction of the 577 Null Mutant of M. tuberculosis

The 577 null mutant Mtb was derived from Mtb H37Rv using the methods described by Parish et al., “glnE Is An Essential Gene in Mycobacterium tuberculosis,” J. Bacteriol. 182(20): 5715-5720 (2000) (which is hereby incorporated by reference in its entirety), which involved four sequential cloning steps and two stage selection after transformation with the suicide vector, p2NIL/5f/hyg/3fVhyg/PacI cassette. First, the 5′ flank region of Rv0577, the 3′ flank region of Rv0577, and the hygromycin resistance marker gene were cloned into p2NIL, and transformed into competent E. coli DH5α. The 5′ flank (5f, 2252 bp) was PCR amplified using the primer pairs: forward, 5′-ATT AAA GCT TAC CCG ACC G CGT GAC CAG CGG TC-3′ (SEQ ID NO: 3), and reverse, 5′-ATT ATC TAG AGA TCA TCC TTT CGT TAG GTG GCG-3′ (SEQ ID NO: 4), and ligated into the p2NIL vector between HindIII/XbaI creating the pNIL5f plasmid. The 3′ flank (3f, 1839 bp) was PCR amplified using primer pairs: forward, 5′-ATT ATC TAG ACA GCA ATA GGG AGC ATC CCG GG-3′ (SEQ ID NO: 5), and reverse, 5′-ATT AGC AGC GAC GGT GTC AAC GGT TC-3′ (SEQ ID NO: 6), and ligated into the XbaI and KpnI sites of the pNIL5f plasmid creating the pNIL5f3fplasmid. The hygromycin^{R} gene was PCR amplified from pMS2 using primer pairs: forward, 5′-ATT ATC TAG ACC CTG TGA ATA GAG GTC CGC-3′ (SEQ ID NO: 7), and reverse, 5′-ATT TCT AGA CTG GAG GAG ATG ATC GAG GAT-3′ (SEQ ID NO: 8), and ligated into the single XbaI site in the middle of 5′ and 3′ flanking sequences, creating the pNIL/5f/hyg/3fplasmid. All PCR products were confirmed by DNA sequence analysis using an automated sequencer (ABI Prism 310 Genetic Analyzer, Perkin Elmer). The PacI cassette from the pGOAL17 plasmid which contains an Ag85 promoter driving the lacZ gene (P_{Ag85}-LacZ) and hsp60 driving the sacB gene (Pₕₛₚ₆₀-sacB) was excised and cloned into a single PacI site within the p2NIL/5f/hyg/3f plasmid to yield the suicide vector, p2NIL/5f/hyg/3f/hyg/PacI cassette (Parish et al., “glnE Is An Essential Gene in Mycobacterium tuberculosis,” J. Bacteriol. 182(20): 5715-5720 (2000), which is hereby incorporated by reference in its entirety). The plasmids obtained from E. coli DH5a clones were confirmed to possess the ˜11 kb fragment. The selection for 577 null mutant Mtb was as follows. Competent MtbH37Rv were transformed by electroporation with the p2NIL5f/hyg/3f/PacI vector, plated onto 7H11 plates containing hygromycin and kanamycin and incubated for 3 weeks. Mtb colonies obtained by this selection step are single crossovers. Next, single colonies were inoculated into hygromycin containing liquid medium and incubated for 2 weeks to select for the second cross-over. Lastly, growing bacteria were plated onto 7H11 agar containing hygromycin, X-gal (˜40 μg/ml) and 2% sucrose for 3 weeks to select for white colonies that have lost the PacI cassette and replacement of the Rv0577 gene by the hygromycin^{R} gene.

### Example 4

Complementation of 577 Null Mutant M. tuberculosis

The 577 null mutant Mtb was genetically complemented by introducing a wild type copy of the Rv0577 gene using the pMSG plasmid vector. The complete coding sequence of Rv0577 gene was PCR amplified from H37Rv genomic DNA using the primer pairs: forward, 5′-ATA TTA ATT AAG ATG CCC AAG AGA AGC-3′ (SEQ ID NO: 9), and reverse, 5′-ATT GGA TCC CTA TTG CTG CGG TGC GG-3′ (SEQ ID NO: 10). The amplicon was cloned into the pMSG vector between the PacI and BamHI restriction sites. The glutamate synthase (GS) promoter PCR amplified from MtbH37Rv using the primer pairs forward (Glute5), 5′-GGA CTA GTG CGA TCA GCC AGT CGA TCA GCA GAG-3′ (SEQ ID NO: 11), and reverse (Glute3) 5′-CCT TAA TTA ATT CCG TCA CAG AAT GCT CCT TTA C-3′ (SEQ ID NO: 12), was cloned into the promoterless pMS2 vector at the SpeI and PacI site(s) generating the pMSG vector (Harth et al., “Expression and Efficient Export of Enzymatically Active Mycobacterium tuberculosis Glutamine Synthetase in Mycobacterium smegmatis and Evidence that the Information for Export is Contained Within the Protein,” J. Biol. Chem. 272: 22728-22735 (1997); Kaps et al., “Energy Transfer Between Fluorescent Proteins Using a Co-Expression System in Mycobacterium smegmatis,” Gene 278: 115-124 (2001); Ehrt et al., “Reprogramming of the Macrophage Transcriptome in Response to Interferon-gamma and Mycobacterium tuberculosis: Signaling Roles of Nitric Oxide Synthase-2 and Phagocyte Oxidase, J Exp Med 194(8):1123-40 (2001), which are hereby incorporated by reference in their entirety). In the pMSG plasmid, the Rv0577 gene under the control of the GS promoter is transcribed constitutively. The pMSG.577 plasmid purified from E. coli was electroporated into competent 577 null mutant Mtb. Transformants were obtained after plating on supplemented 7H11 plates containing 25 μg/ml kanamycin. Complementation was confirmed by Southern blot, PCR amplification, and Western blot analyses.

### Example 5

Over-Expression of Rv0577 in M. smegmatis

The Rv0577 gene was PCR amplified with primers containing PacI and HindIII enzyme cleavage sites for subsequent cloning using the following primers: forward, 5′-CCC TTA AAT GTC CGC CAC CTA ACG AAA G-3′) (SEQ ID NO: 13) and reverse (5′-CCC AAG CTT CTA GCA TTC TCC GAA-3′ (SEQ ID NO: 14) primers which amplified the coordinates 671137 to 672002 of the full Rv genome (GenBank Accession No. NC_{—}000962). The amplicon sequenced validated as Rv0577 was cloned into pMS3 a plasmid derived from the promoterless pMS2 vector by insertion of the M. tuberculosis heat shock protein promoter (hsp6o) in front of the multiple cloning sites (Kaps et al., “Energy Transfer Between Fluorescent Proteins Using a Co-Expression System in Mycobacterium smegmatis,” Gene 278: 115-124 (2001); Ehrt et al., “Reprogramming of the Macrophage Transcriptome in Response to Interferon-gamma and Mycobacterium tuberculosis: Signaling Roles of Nitric Oxide Synthase-2 and Phagocyte Oxidase, J Exp Med 194(8):1123-40 (2001), which are hereby incorporated by reference in their entirety). The pMS3 (pMS2) plasmid also contains the hygromycin^{R} resistance gene. The plasmids, pMS3 and pMS3.577 purified from E. coli, respectively DH5α and JM109, were transformed into competent M. smegmatis MC²155. The transformants plated on supplemented 7H11 agar containing hygromycin (50 μg/ml) were validated as transformants containing pMS3 or pMS3.577 by analysis of the amplicon size after PCR amplification.

For analysis of the genetic constructs, gene amplification by PCR utilized primer pairs to detect all mycobacteria, as follows: 16S rRNA: forward, 5′-ACG GTG GGT ACT AGG TGT GGG TTT C-3′ (SEQ ID NO: 15) and reverse, 5′-TCT GCG ATT ACT AGC GAC TCC GAC TTC A -3′ (SEQ ID NO: 16); only Mtb complex subspecies (MPB70) forward, 5′-GGC GAT CTG GTG GGC CCG -3′ (SEQ ID NO: 17), and reverse, 5′-CGC CGG AGG CAT TAG CAC GCT -3′ (SEQ ID NO: 18); only M. smegmatis (MsO9l 1) forward, 5′-ACG CGA AGT CGG GCA ACA C 3′ (SEQ ID NO: 19) and reverse, 5′-GCG GCA GCG GGC GGG AGC AAC T -3′ (SEQ ID NO: 20), designed using Tigr.org database); and Rv0577 (cfp32) forward, 5′-ATG CCC AAG AGA AGC GAA TAC AGG CAA-3′ (SEQ ID NO: 21), and reverse 5′-CTA TTG CTG CGG TGC GGG CTT CAA-3′ (SEQ ID NO: 22). Additional primer pairs were as follows for: pMS3 multiple cloning sites (MCS) forward, 5′-CGA GGG GAT TAC ACA TGA CCA ACT-3′ (SEQ ID NO: 23) and reverse, 5′-CGG AAG AGC GCC CAA TAC G-3′ (SEQ ID NO: 24); pMSG kan-flag-MCS sequencing: forward, 5′-ATA ACG TTC TCG GCT CGA TGA TCC-3′ (SEQ ID NO: 25), and reverse, 5′-ATC CCC TGA TTC TGT GGA TAA CCG TAT TA-3′ (SEQ ID NO: 26); and Rv0577 sequencing forward, 5′-ACC ACC TTG TCC ACC ACC GCA T-3′ (SEQ ID NO: 27), and reverse, 5′-CGA ATC ATT GGC ACG TCT ACT TTG-3′ (SEQ ID NO: 28). The Rv0577 deletion locus of 577 Null was PCR amplified using the following primer pairs: forward 577PROF, 5′-GTG GCT TGG CGG GCA CGG TGG AG-3′ (SEQ ID NO: 29) and reverse lDn577R, 5′-GTG GCA CCG GCG GCA CCG CAC ACC T-3′ (SEQ ID NO: 30).

### Example 6

Selective Induction of IL-10 in M. tuberculosis

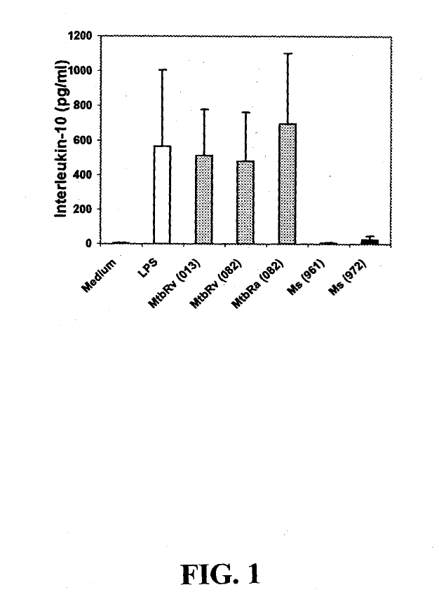

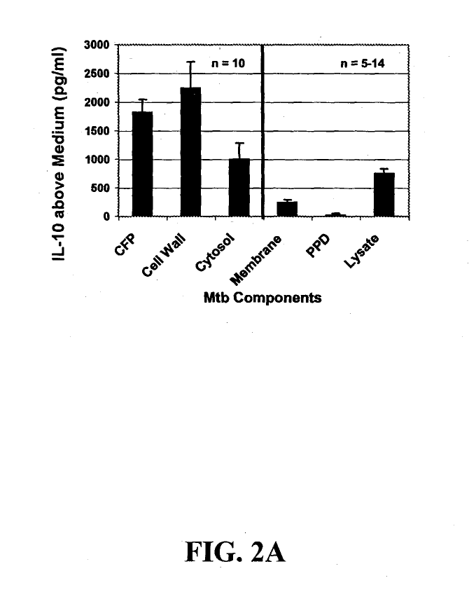

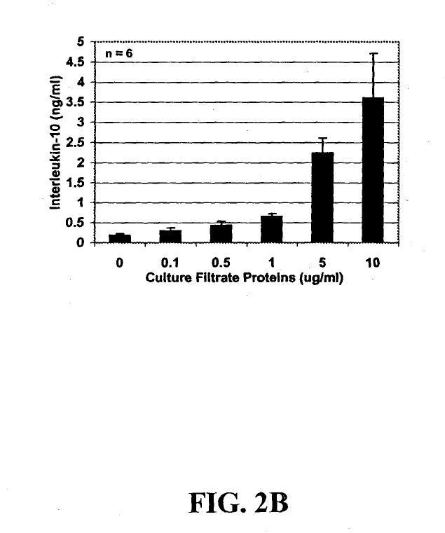

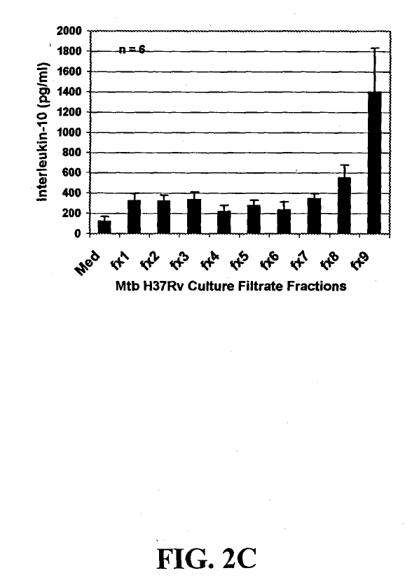

To evaluate whether IL-10 induction is restricted to Mtb, a comparison to M smegmatis, a non-pathogenic mycobacteria, was conducted. Mtb H37Rv (virulent laboratory strain) isolates and Mtb H37Ra (an attenuated strain) infection of human monocytes induced IL-10 production at levels that were similar to LPS. These results are shown in FIG. 1. M bovis BCG also induced IL-10. In contrast, two strains of M smegmatis did not induce IL-10. No reduction in monocyte viability was seen in the M smegmatis-infected monocytes at the experiments' end. To discern what component of Mtb bore IL-10 inducing activity, the whole cell lysate, culture filtrate (CFP), cell wall, cytosol, and cell membrane fractions were evaluated and compared with purified protein derivative (PPD) of Mtb. Significant IL-10 inducing activity resided in the cell wall, cytosol, and CFP and, to a lesser degree, the whole cell lysate, as shown in FIG. 2A. The CFP was of greatest interest as it is known to contain proteins that are immunogenic and are perfectly situated for immunomodulation. These properties of CFP led to its further evaluation. The stimulation of fresh monocytes by CFP to produce IL-10 was dose-dependent (from 0.1 to 10 μg/ml), as shown in FIG. 2B. Proteinase K treatment (5 μl, overnight 4° C.) of 0.1 μg CFP reduced 80% of the IL-10 inducing activity while IL-10 production by fresh monocytes treated with 0.1 μg/ml CFP was 235±39 pg/ml (n=3). L-10 production by monocytes cultured with proteinase K or in medium alone was similarly low (n =3). As shown in FIG. 2C, the IL-10 inducing activity could be enriched by anion-exchange chromatography. Compared to 1 tg/ml CFP, anion exchange chromatography fractions (fx1 to fx9) at a concentration of 0.1 μg/ml were tested, and the highest IL-10 inducing activity was seen in fx9. The IL-10 induction by 0.2 μg/ml of fx9 of CFP was comparable to 5 μg/ml of whole CFP, as seen in FIG. 2B versus FIG. 2C. A prominent ˜32 kDa protein was seen most prominently in fx9 on silver stained gel of this fraction, and was extracted to clone CFP32, the protein encoded by Rv0577. In Table 1, below, a sandwich ELISA was used to correlate the amount of Rv0577 (expressed as pg Rv0577 per μg of reagent) with IL-10 inducing activity (expressed as pg IL-10 per μg reagent).

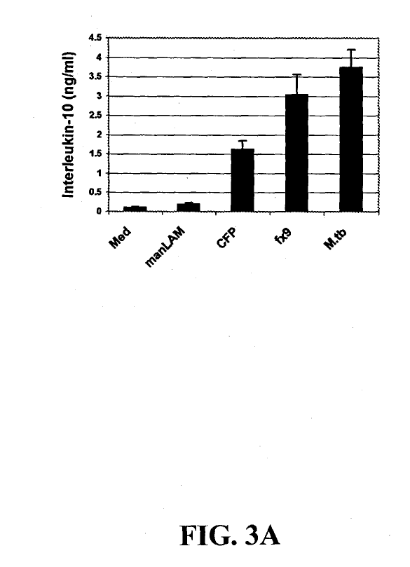

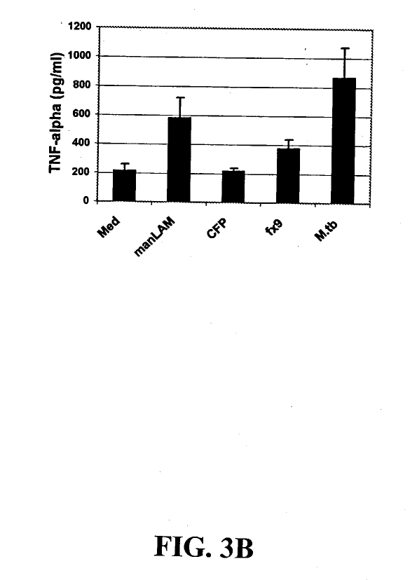

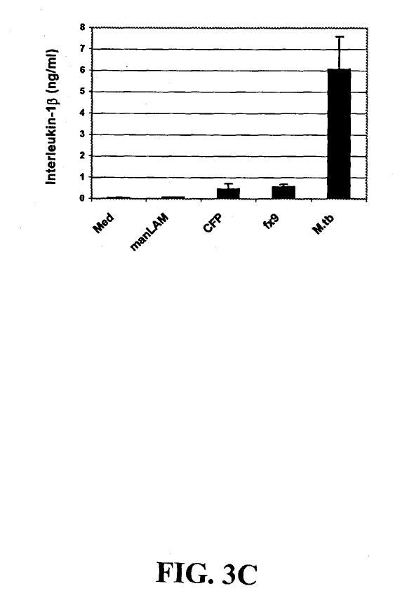

TABLE 1
Assessment of Rv0577 Protein (CFP32) in Mtb Reagents
Tested for IL-10 Inducing Activity¹

| | Amount of Rv0577 | IL-10 |
|---|---|---|
| | protein Activity | Inducing |
| Mtb reagent | (pg Rv05774/μg reagent) | (pg IL-10/μg reagent) |
| manLAM | 28 ± 21 | 82 ± 44 |
| PPD | 53 ± 37 | 51 ± 28 |
| Whole Cell | 1559 ± 378 | 759 ± 83 |
| Lysate | | |
| Cytosol | 1043 ± 66 | 1008 ± 280 |
| CFP (lot1) | 663 ± 195 | 1630 ± 225 |
| Fraction 9 | 61,000 ± 5,300 | 15,210 ± 2,600 |
| of CFP (f × 9) | | |
| 1The ELISA assay for quantitation of Rv0577 protein in Mtb reagents was performed at least twice and is expressed as mean ± SD. The production of IL-10 induced by each reagent was by the following number of donors: 12, 5, 14, 10, 17 and 10, respectively. The amount of each reagent used are indicated in the legend of Figures 2 and 3. All Mtb reagents shown were tested for endotoxin (lipopolysaccharide, LPS) and contained less than 50 pg of LPS per μg of reagent. | | |
| #In this system, a minimum of 10 ng/ml of LPS is needed to induce IL-10; and 100 ng/ml is required to attain IL-10 levels comparable to 0.5 cfu Mtb. | | |

The data presented in Table 1 suggest a positive correlation between CFP32 and IL-10 inducing activity. Whether IL-10 is produced in concert with pro-inflammatory cytokines was examined next. In addition to IL-10, Mtb induces the simultaneous production of high levels of both IL-1β and TNF-α. The major component of Mtb cell wall, mannose capped lipoarabinomannan (manLam), induced only TNF-α. In contrast, CFP and an anion exchange chromatography fraction (fx9) of CFP induced minimal amounts of IL-1β and TNF-α, while possessing high IL-10 inducing activity, as shown in FIGS. 3A-C. These data suggest a more selective induction of cytokines by CFP and CFP fx9 of Mtb. The more selective induction of IL-10 by CFP and fx9 while Mtb and LPS simultaneously induce all three cytokines (IL-i 0, TNF-α and IL-1β) argues against contamination of CFP and fx9 of Mtb by LPS or other microorganisms.

### Example 7

M. tuberculosis Encodes a Protein Conferring IL-10 Inducing Activity

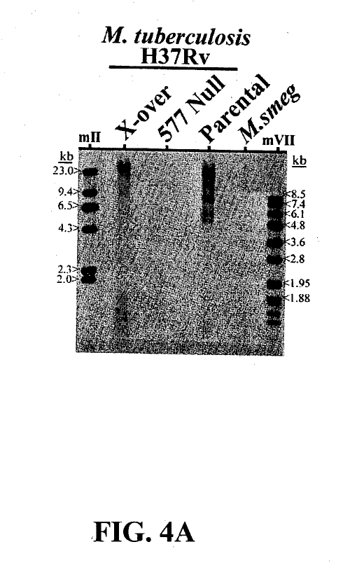

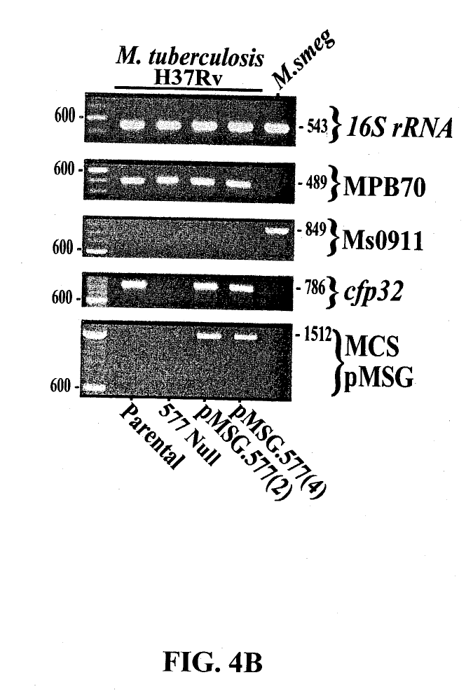

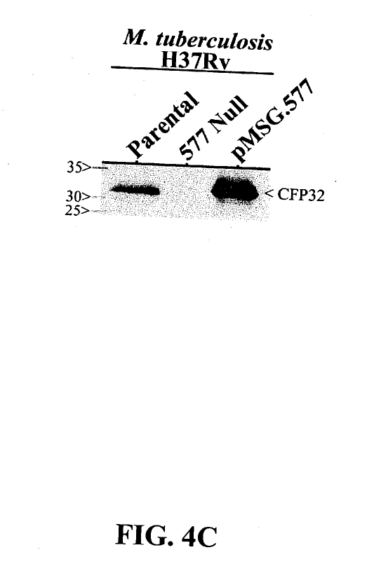

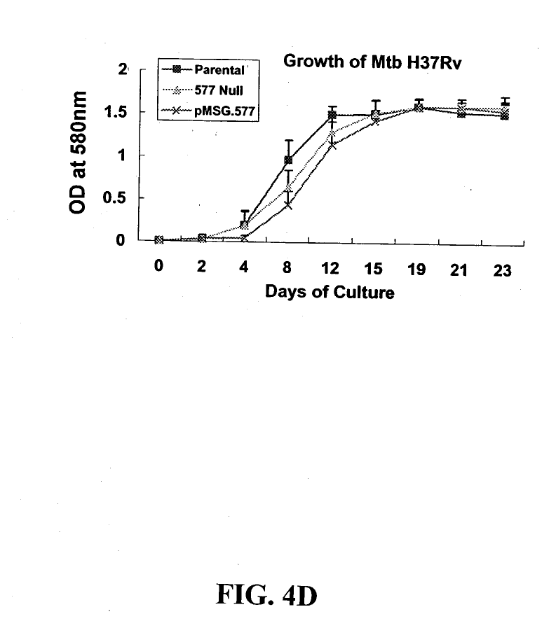

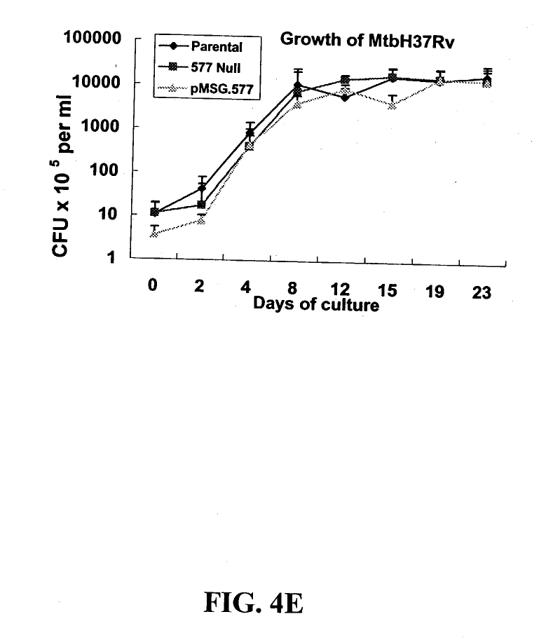

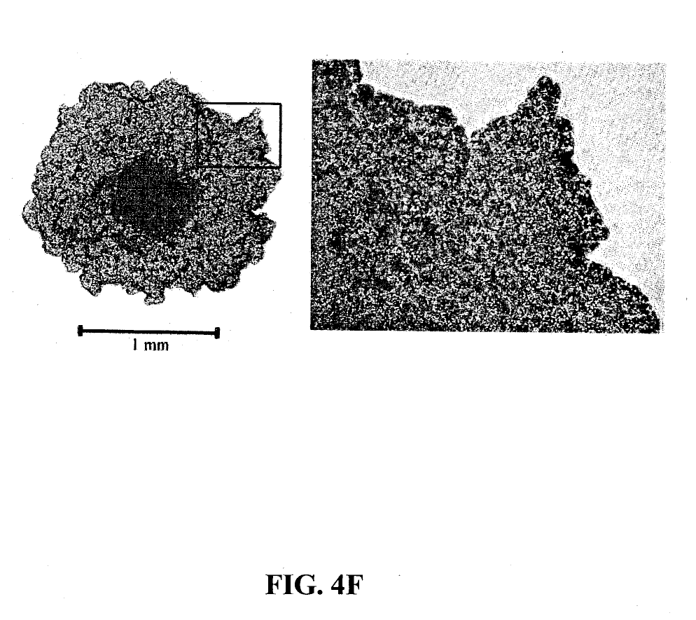

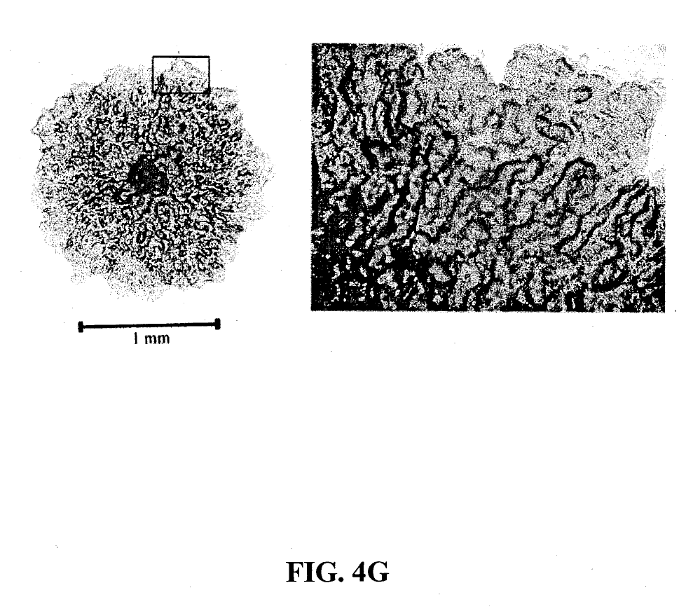

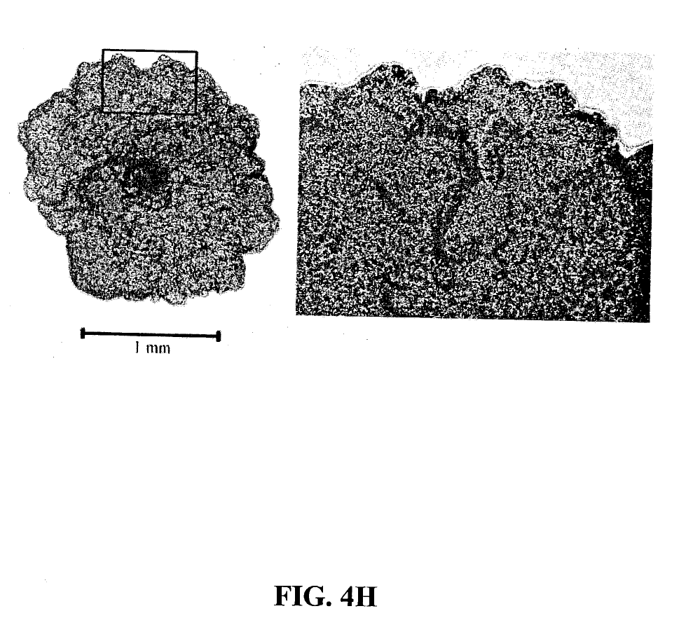

To show that Rv0577 encodes a protein that conferred IL-10 inducing activity to Mtb, a 577 null mutant was created by homologous recombination to replace Rv0577 with the hygromycin B resistance gene. Southern blot analysis demonstrated the step-wise derivation of the 577 null (double cross-over, replacement of Rv0577) from the single cross-over (X-over) which still retains the Rv0577 gene. As internal controls, the presence of Rv0577 in the parental MtbH3 7Rv and absence of Rv0577 in M. smegmatis are illustrated in FIG. 4A. PCR gene amplification analysis demonstrate the presence of Rv0577 within the pMSG plasmid of the complemented 577 null mutant Mtb and confirmed the loss of Rv0577 in the 577 null mutant, as shown in FIG. 4B. Using Western blot analysis, the complementation of Rv0577 with pMSG.577 plasmid under the constitutive glutamine synthase promoter was capable of restoring protein expression and the 577 null mutant lacked CFP32 expression, shown in FIG. 4C. The growth of the 577 null mutant and complemented null mutant Mtb in liquid and solid medium was examined next. Growth of the parental and mutant isolates were quantitated by absorbance measurement and by plating of individual colonies on agar. The 577 null mutant compared to parental Mtb showed similar growth kinetics, shown in FIG. 4D and FIG. 4E. Compared to parental and 577 null Mtb, the initial proliferation of the complemented 577 null mutant was slightly slower but the difference was erased by 2 weeks of culture. Evaluation of the colony morphology grown 7H11 enriched agar showed that the 577 null mutant (gene knockout), shown in FIG. 4G, differed from that of the parental (wildtype) Mtb, shown in FIG. 4F. The parental had a smooth topography and colony edges, while the Rv0577 null mutant had a much more mountainous topography and ruffled edges. This phenotype was restored by complementation with the pMSG.577 plasmid, as shown in FIG. 4H.

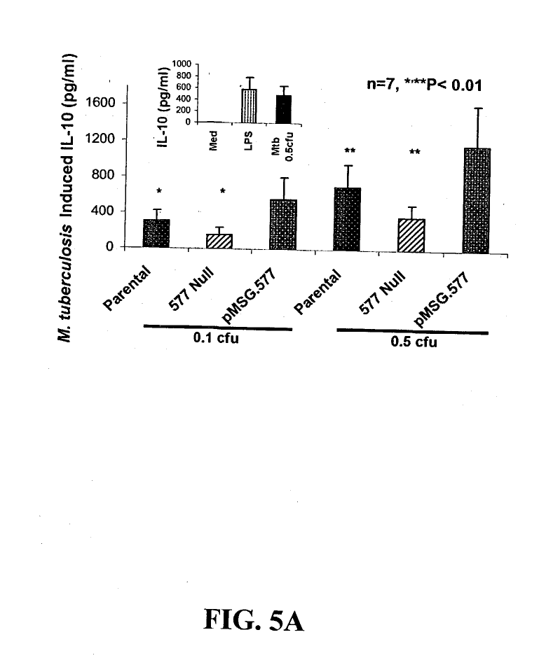

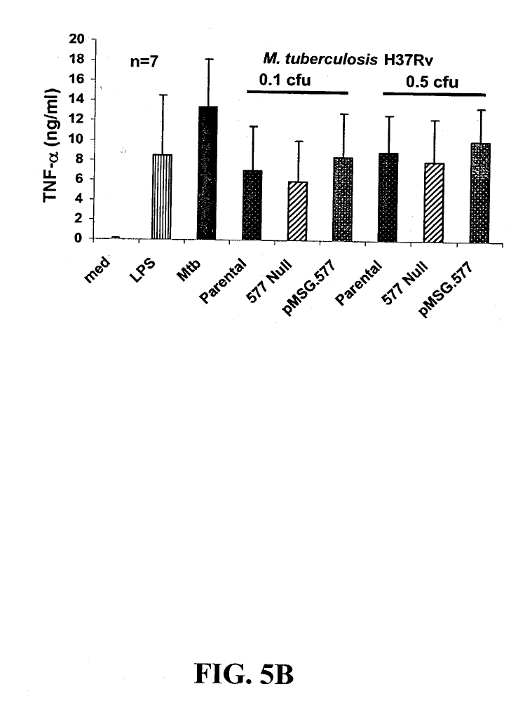

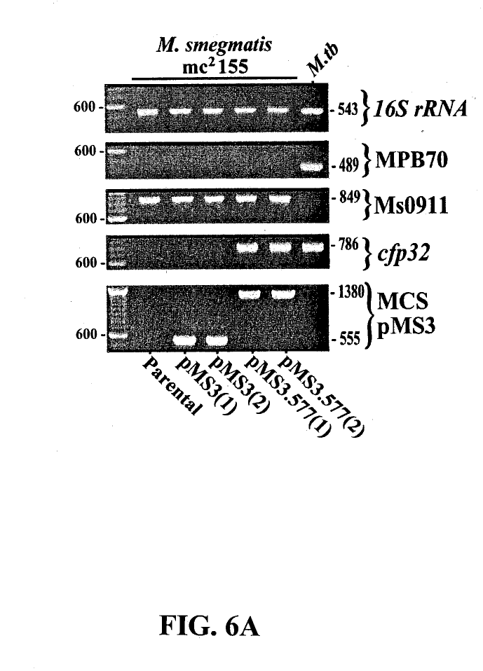

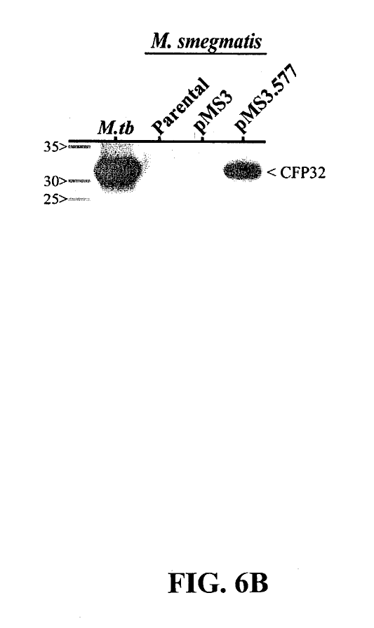

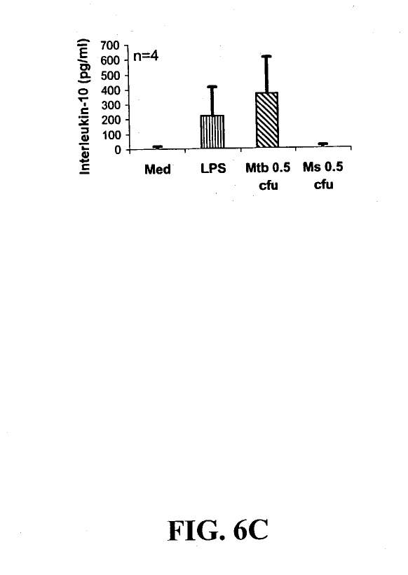

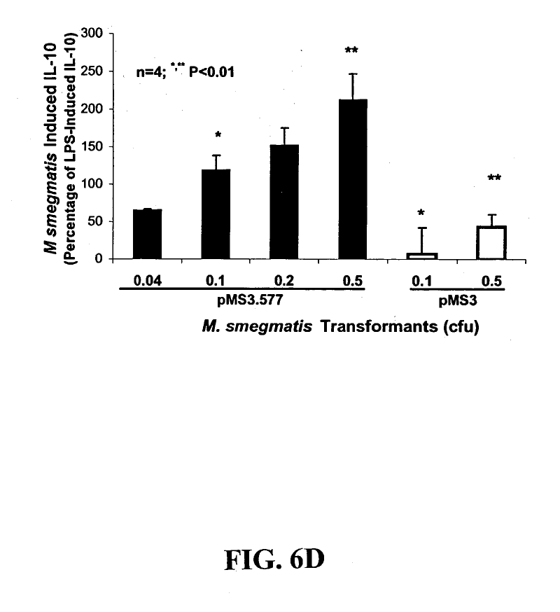

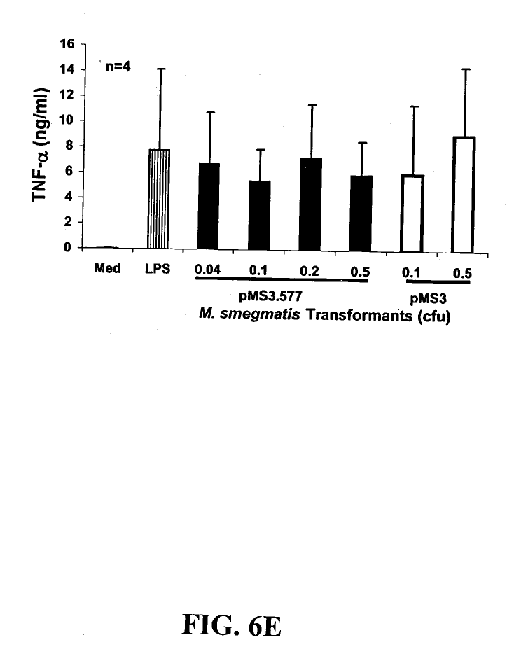

The availability of these mutants allowed the testing of whether the expression of Rv0577 is associated with IL-10 production. As illustrated in FIG. 5A, the Rv0577 null mutant compared to parental Mtb showed ≧50% reduction in IL-10 inducing activity that was statistically significant for both doses tested, 0.1 cfu/Mφ and for 0.5 cfu/Mφ (p≦0.01). Furthermore, complementation of the 577 null mutant restored IL-10 inducing activity. In contrast to IL-10, similar amounts of TNF-α were produced by monocytes infected with parental, 577 null, and complemented 577 null mutant Mtb, shown in FIG. 5B. To further validate that Rv0577 encodes a protein that conferred IL-10 inducing activity, M. smegmatis was transformed with pMS3.577 or pMS3, both expressing the hygromycin B resistance gene. Gene amplification analysis showed that M. smegmatis transformed with pMS3.577 contained the Rv0577 within the pMS3 cloning site, as shown in FIG. 6A. Furthermore, pMS3.577 M. smegmatis expressed Rv0577 protein. In contrast, neither parental M. smegmatis nor pMS3 M. smegmatis had detectable expression of Rv0577 protein, shown in FIG. 6B. As shown in FIG. 6C, parental M. smegmatis infection of human monocytes induced little to no IL-10 above medium, while LPS and MtbH37Rv, internal controls for each experiment, induced high amounts of IL-10 that showed considerable donor to donor variability. Because of the donor variability in IL-10 production, the induction of IL-10 by M. smegmatis transformants was expressed as a percentage of LPS-induced IL-10, as shown in FIG. 6D. Phenotypically, over-expression of Rv0577 by M. smegmatis led to a dose-dependent induction of IL-10 production, FIG. 6D. The induction of IL-10 is statistically significantly higher in M. smegmatis transformed with pMS3.577 than pMS3, as shown in FIG. 6D. Moreover, infection by 0.5 cfu of M. smegmatis transformed with pMS3.577 resulted in levels of IL-10 production comparable to same inoculum of Mtb, as shown in FIG. 6C versus 6D. M. smegmatis transformed with pMSG.577 (the plasmid used to complement the 577 null Mtb mutant) similarly conferred the ability to induce IL-10(n=2). The specificity of the effect of Rv0577 protein expression on IL-10 induction is further suggested by the finding that TNF-α production was similar upon monocytes infection by either pMS3.577 or pMS3 transformed M. smegmatis, as shown in FIG. 6E.

M. tuberculosis, in contrast to M. smegmatis infection of human monocytes, triggered the production of IL-10, a potent suppressor of antimicrobial activity. The screening of the culture filtrate of Mtb H37Rv for a factor with IL-10 inducing activity identified Rv0577. Through Rv0577 gene knockout and complementation in Mtb, it was demonstrated that 577 null mutant Mtb infection of monocytes produced significantly less amounts of IL-10, and complementation of the 577 null mutant restored the expression of Rv0577 (CFP32) and IL-10 production to levels comparable to parental Mtb. From the 577 null mutant monocyte infection studies, the attributed IL-10 inducing activity to Rv0577 is in the order ˜60%. Moreover, in a heterologous mycobacteria, M. smegmatis, over-expression of Rv0577 by another plasmid resulted in an inoculum-dose dependent IL-10production by infected monocytes. The changes observed with IL-10 production associated with the molecular manipulation of Rv0577 were specific because the simultaneous measurement of TNF-A produced by monocytes infected with M. tuberculosis or M. smegmatis mutants showed no significant differences.

The cytokine milieu as microbes encounter the innate and acquired immune cells is thought to be critical to the overall immune response and effective elimination of the invading agent (Medzhitov et al., “Innate Immunity,” N Engl J Med. 343(5):338-44 (2000); Sieling et al., “Toll-Like Receptors: Mammalian “Taste Receptors” For a Smorgasbord of Microbial Invaders,” Curr Opin Microbiol 5(1):70-5 (2002); Janeway et al., “Innate Immune Recognition,” Annu Rev Immunol 20:197-216 ( 2002); Fitzgerald et al., “The Role of the Interleukin-1/Toll-Like Receptor Superfamily in Inflammation and Host Defense,” Microbes Infect. 2(8):933-43 (2000); which are hereby incorporated by reference in their entirety). Manipulation of this environment is thought to be a strategy for microbial evasion and survival, and several examples have been reported. One strategy, for example, is the interference with the production of TNF-α and IFN-λ, cytokines critical for innate and acquired immune cell activation to clear intracellular microbes. The Yersinia Yop gene product blocks TNF-α production by macrophages through interference with intracellular signaling molecules (Orth et al., “Disruption of Signaling by Yersinia Effector YopJ, a Ubiquitin-Like Protein Protease.,” Science 290(5496): 1594-7 (2000); Boland et al., “Role of YopP in Suppression of Tumor Necrosis Factor Alpha Release by Macrophages During Yersinia Infection,” Infect. Immun. 66(5):1878-84 (1998); Cornelis et al., “Yersinia Lead SUMO Attack,” Nat. Med. 7:21-23 (2001), which are hereby incorporated by reference in their entirety). The adenovirus E3 14.7 Kd protein is reported to interfere with TNF-α mediated cytolytic activity, thereby blocking viral clearance (Trufariello et al., “Adenovirus E3 14.7-kDa Protein, an Antagonist of Tumor Necrosis Factor Cytolysis, Increases the Virulence of Vaccinia Virus in Severe Combined Immunodeficient Mice,” Proc. Natl. Acad. Sci. USA 91:10987-91 (1994); Trufariello et al., “Adenovirus E3 14.7-kDa Protein, an Antagonist of Tumor Necrosis Factor Cytolysis, Increases the Virulence of Vaccinia Virus in a Murine Pneumonia Model,” J. Virol. 68:453-62 (1994), which are hereby incorporated by reference in their entirety). The Mtb cell wall constituent, mannosylated lipoarabinomannans (manLAM), is reported to suppress IL-12 production, a cytokine required for T helper-1 cell maturation and secretion of IFN-λ (Nigou et al., “Mannosylated Lipoarabinomannans Inhibit IL-12 Production by Human Dendritic Cells: Evidence for a Negative Signal Delivered through the Mannose Receptor,” J. Immunol. 166(12):7477-85 (2001), which is hereby incorporated by its entirety). Encoding or inducing the production of factors to suppress host immune response is another strategy (Stockl et al., “Human Major Group Rhinoviruses Down-Modulate the Accessory Function of Monocytes by Inducing IL-10,” J. Clin. Invest. 104(7):957-65 (1999); Fleming et al., “A Homolog of Interleukin-10 is Encoded by the Poxvirus Orf Virus,” J. Virol. 71(6):4857-61 (1997); Vockerodt et al., “The Epstein-Barr Virus Latent Membrane Protein 1 Induces Interleukin-10 in Burkitt's Lymphoma Cells but not in Hodgkin's Cells Involving the p38/SAPK2 Pathway,” Virology 280(2):183-98 (2001); Henke et al., “Viral IL-10 Gene Transfer Decreases Inflammation and Cell Adhesion Molecule Expression in a Rat Model of Venous Thrombosis,” J. Immunol. 164(4):2131-41 (2000); Suzuki et al., “Viral Interleukin 10 (IL-10), the Human Herpes Virus 4 Cellular IL-10 Homologue, Induces Local Anergy to Allogeneic and Syngeneic Tumors,” J. Exp. Med. 182(2):477-86 (1995), which are hereby incorporated by reference in their entirety). The EBV encoded human IL-10 homolog and the EBV latent protein-1 that elicits IL-10 production both facilitate viral survival and pathogenesis through IL-10's immune suppressive activity (Vockerodt et al., “The Epstein-Barr Virus Latent Membrane Protein 1 Induces Interleukin-10 in Burkitt's Lymphoma Cells but not in Hodgkin's Cells Involving the p38/SAPK2 Pathway,” Virology 280(2):183-98 (2001); Henke et al., “Viral IL-10 Gene Transfer Decreases Inflammation and Cell Adhesion Molecule Expression in a Rat Model of Venous Thrombosis,” J. Immunol. 164(4):2131-41 (2000); Suzuki et al., “Viral Interleukin 10 (IL-10), the Human Herpes Virus 4 Cellular IL-10 Homologue, Induces Local Anergy to Allogeneic and Syngeneic Tumors,” J. Exp. Med. 182(2):477-86 (1995), which are hereby incorporated by reference in their entirety). Induction of host IL-10 by Schistosoma eggs and HIV-1 gp41 and gp120 is thought to contribute to the immune suppression caused by these pathogens (Wynn et al., “Analysis of Granuloma Formation (by Schistomsoma eggs) in Double Cytokine-Deficient Mice Reveals a Central Role for IL-10 in Polarizing Both T Helper Cell 1- and T Helper Cell 2-Type Cytokine Responses In vivo,” J. Immunol. 159(10):5014-23 (1997); Barcova et al., “gp41 Envelope Protein of Human Immunodeficiency Virus Induces Interleukin (IL)-10 in Monocytes, but not in B, T, or NK Cells, Leading to Reduced IL-2 and Interferon-Gamma Production,” J. Infect. Dis. 177(4):905-13 (1998); Taoufik et al., “Human Immunodeficiency Virus gp120 Inhibits Interleukin-12 Secretion by Human Monocytes: an Indirect Interleukin-10-Mediated Effect,” Blood 89(8):2842-8 (1997); Koutsonikolis et al., “HIV-1 Recombinant gp41 Induces IL-10 Expression and Production in Peripheral Blood Monocytes but not in T-Lymphocytes,” Immunol. Lett. 55(2):109-13 (1997); Schols et al., “Human Immunodeficiency Virus Type 1 gp120 Induces Anergy in Human Peripheral Blood Lymphocytes by Inducing Interleukin-10 Production,” J. Virol. 70(8):4953-60 (1996), which are hereby incorporated by reference in their entirety). In animal models, IL-10 has been shown in mice to enhance microbe survival because depletion of IL-10 increased resistance to Candida infection (Tavares et al., “Increased Resistance to Systemic Candidiasis in Athymic or Interleukin-10-Depleted Mice,” J. Infect. Dis. 182(1):266-73 (2000), which is hereby incorporated by reference in its entirety). Moreover, in murine leishmaniasis, a model of Th1 and Th2 immune modulation that is linked to disease outcome, IL-10R blockage by itself can induce near-cure (Murray et al., “Interleukin-10 (IL-10) in Experimental Visceral Leishmaniasis and IL-10 Receptor Blockage as Immunotherapy,” Infect. Immun. 70:6284-6293 (2002), which is hereby incorporated by reference in its entirety). Furthermore, in subsets of patients with active Th studied, anti-IL-10 Ab has been shown to improve immunity against Mtb, and these patients also have IL-10 producing T cell clones (Gong et al., “Interleukin-10 Downregulates Mycobacterium tuberculosis-Induced Th1 Responses and CTLA-4 Expression,” Infect. Immun. 64(3) :913-8 (1996); Mendez-Samperio et al., “Depletion of Endogenous Interleukin-10 Augments Interleukin-1 Beta Secretion by Mycobacterium bovis BCG-Reactive Human Cells,” Clin. Diagn. Lab. Immunol. 4(2):138-41 PMID: 9067646 (1997); Baliko et al., “Th2 Biased Immune Response in Cases with Active Mycobacterium tuberculosis Infection and Tuberculin Anergy,” FEMS Immunol. Med. Microbiol. 22(3):199-204 (1998); Boussiotis et al., “IL-10-Producing T Cells Suppress Immune Responses in Anergic Tuberculosis Patients,” J. Clin. Invest. 105(9):1317-25 (2000), which are hereby incorporated by reference in their entirety). These data from other infectious agents, coupled with the demonstrated role of IL-10 in modulating tissue bacilli burden and pathology of murine M. bovis infection and the Th patient data, strongly suggest that IL-10 plays a role in M. tuberculosis infection and disease. The identification of Rv0577 encoding a protein that induces human monocyte production of IL-10 extends the knowledge of one strategy of M. tuberculosis for survival within man that acts by immune suppression.

There is data that shows that only M. tuberculosis subspecies members possess Rv0577 and that Rv0577 is absent from all non-tuberculosis mycobacteria examined thus far. The major subspecies members of Mtb are M. bovis Bacillus Calmette-Guerin (BCG), M. africanum, and M. microti.

In addition, in over 70 M tuberculosis clinical isolates examined thus far, including representative IS6110 “genotypes” , all possess the Rv0577 gene, and all M. tuberculosis clinical isolates examined thus far express CFP32. Furthermore, CFP32 has been detected in induced sputum of patients with active pulmonary Tb but not from patients with other lung diseases, and importantly, CFP32 levels were positively correlated with IL-10 but not with IFN-γ. These data minimally suggest that Rv0577 is present at the site of human disease, and the association of CFP32 with local IL-10 production is intriguing and implicate a potential role in disease. The finding that the expression of CFP32 (Rv0577) correlates with IL-10 production by human monocytes infected with molecular constructs of M. tuberculosis and M. smegmatis provides evidence for co-opting IL-10 as a mechanism for inuunomodulation by M. tuberculosis. This is a potentially new paradigm for M. tuberculosis to evade immune elimination.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A nucleic acid construct comprising:
a nucleic acid molecule that encodes a factor suppressing an immune response to Mycobacterium tuberculosis in a host subject, wherein the nucleic acid molecule either: 1) has a nucleotide sequence corresponding to SEQ ID NO: 1; 2) has a nucleotide sequence that hybridizes to the nucleic acid corresponding to SEQ ID NO: 1 under stringent conditions characterized by a hybridization buffer comprising 5×SSC at a temperature of 54° C.; 3) is at least 55% similar to the nucleotide sequence of SEQ ID NO: 1 by basic BLAST using default parameters analysis; or 4) encodes a protein or polypeptide having an amino acid sequence corresponding to SEQ ID NO: 2;
an operably linked DNA promoter; and
an operably linked 3′ regulatory region.

2. The nucleic acid construct according to claim 1, wherein the nucleic acid molecule has a nucleotide sequence corresponding to SEQ ID NO: 1.

3. The nucleic acid construct according to claim 1, wherein the nucleic acid molecule has a nucleotide sequence that hybridizes to the nucleic acid corresponding to SEQ ID NO: 1 under stringent conditions characterized by a hybridization buffer comprising 5×SSC at a temperature of 54° C.

4. The nucleic acid construct according to claim 1, wherein the nucleic acid molecule is at least 55% similar to the nucleotide sequence of SEQ ID NO: 1 by basic BLAST using default parameters analysis.

5. The nucleic acid construct according to claim 1, wherein the nucleic acid molecule encodes a protein or polypeptide having an amino acid sequence corresponding to SEQ ID NO: 2.

6. The nucleic acid construct according to claim 1, wherein the DNA promoter is a heterologous promoter.

7. The nucleic acid construct according to claim 1, wherein the DNA promoter is a homologous promoter.

8. An expression vector comprising:
the nucleic acid construct according to claim 1.

9. The expression vector according to claim 8, wherein the nucleic acid molecule is inserted in a proper sense orientation and correct reading frame.

10. A host cell transduced with the expression vector according to claim 9.

11. The host cell according to claim 10, wherein the cell is selected from the group consisting of a bacterial cell, a virus, a yeast cell, and a mammalian cell.

12. A host cell according to claim 11, wherein the host cell is a bacterial cell.

13. An isolated antibody or binding portion thereof against a protein or polypeptide having an amino acid sequence corresponding to SEQ ID NO: 2.

14. An isolated antibody or binding portion thereof according to claim 13, wherein the antibody is monoclonal or polyclonal.

15. The antibody or binding portion thereof according to claim 14, wherein the binding portion thereof is selected from the group consisting of an Fab fragment, an F(ab′)₂ fragment, and an Fv fragment.

16. A method for detection of Mycobacterium tuberculosis specific antibodies in a sample of tissue or body fluids comprising:
providing an isolated protein or polypeptide having an amino acid corresponding to SEQ ID NO: 2 as an antigen;
contacting the sample with the antigen under conditions effective to allow formation of a complex of the antigen bound to antibodies which recognize the antigen; and
detecting if any of the complex is present, thereby indicating a presence of Mycobacterium tuberculosis the sample.

17. The method according to claim 16, wherein said detecting is carried out with an assay system selected from the group consisting of an enzyme-linked immunosorbent assay, a radioimmunoassay, a gel diffusion precipitin reaction assay, an immunodiffusion assay, an agglutination assay, a fluorescent immunoassay, a protein A immunoassay, and an immunoelectrophoresis assay.

18. A method for detection of Mycobacterium tuberculosis in a sample of tissue or body fluids comprising:
providing an antibody or binding portion thereof according to claim 13;
contacting the sample with the antibody or binding portion thereof under conditions effective to allow formation of a complex of the antibody or binding portion thereof and an antigen recognized by the antibody or binding portion thereof; and
detecting if any of the complex is present, thereby indicating a presence of Mycobacterium tuberculosis in the sample.

19. The method according to claim 18, wherein an antibody is used to carry out the method and the antibody is selected from the group consisting of a monoclonal antibody and a polyclonal antibody.

20. The method according to claim 18, wherein a binding portion thereof is used to carry out the method and the binding portion is selected from the group consisting of an Fab fragment, an F(ab′)₂ fragment, and an Fv fragment.

21. The method according to claim 18, wherein said detecting is carried out with an assay system selected from the group consisting of an enzyme-linked immunosorbent assay, a radioimmunoassay, a gel diffusion precipitin reaction assay, an immunodiffusion assay, an agglutination assay, a fluorescent immunoassay, a protein A immunoassay, and an immunoelectrophoresis assay.

22. A method for detection of Mycobacterium tuberculosis in a sample of tissue or body fluids comprising:
providing a nucleic acid molecule according to claim 1 as a probe in a nucleic acid hybridization assay wherein the nucleic acid molecule encodes a factor suppressing an immune response to Mycobacterium tuberculosis in a host subject and either: 1) has a nucleotide sequence corresponding to SEQ ID NO: 1; 2) has a nucleotide sequence that hybridizes to the nucleic acid corresponding to SEQ ID NO: 1 under stringent conditions characterized by a hybridization buffer comprising 5×SSC at a temperature of 54° C.; 3) is at least 55% similar to the nucleotide sequence of SEQ ID NO: 1 by basic BLAST using default parameters analysis; or 4) encodes a protein or polypeptide having an amino acid sequence corresponding to SEQ ID NO: 2.;
contacting the sample with the probe under conditions effective to permit formation of a complex of the probe and nucleic acid which hybridizes to the probe; and
detecting formation of the complex in the sample, thereby indicating a presence of Mycobacterium tuberculosis in the sample.

23. A method for detection of Mycobacterium tuberculosis in a sample of tissue or body fluids comprising:
providing a nucleic acid molecule as a probe or primer in a gene amplification detection procedure, wherein the nucleic acid molecule encodes a factor suppressing an immune response to Mycobacterium tuberculosis in a host subject, and either: 1) encodes a factor suppressing an immune response to Mycobacterium tuberculosis in a host subject; 2) has a nucleotide sequence corresponding to SEQ ID NO: 1; 3) has a nucleotide sequence that hybridizes to the nucleic acid corresponding to SEQ ID NO: 1 under stringent conditions characterized by a hybridization buffer comprising 5×SSC at a temperature of 54° C.; or 4) is at least 55% similar to the nucleotide sequence of SEQ ID NO: 1 by basic BLAST using default parameters analysis;
contacting the sample with the probe or primer under conditions effective to amplify probe or primer-specific nucleic acid molecules; and
detecting any amplified probe or primer-specific molecules, thereby indicating a presence of Mycobacterium tuberculosis in the sample.

24. A method of vaccinating a mammal against infection by Mycobacterium tuberculosis comprising:
administering an effective amount of an isolated protein or polypeptide having an amino acid sequence corresponding to SEQ ID NO: 2 to the mammal.

25. The method according to claim 24, wherein said administering is oral, intradermal, intramuscular, intraperitoneal, intravenous, subcutaneous, or intranasal.

26. A vaccine for preventing infection and disease of mammals by Mycobacterium tuberculosis comprising:
an isolated protein or polypeptide having an amino acid sequence corresponding to SEQ ID NO: 2; and
a pharmaceutically-acceptable carrier.

27. The vaccine according to claim 26, wherein said protein or polypeptide is purified.

28. A method of vaccinating mammals against infection by Mycobacterium tuberculosis comprising:
administering an effective amount of the vaccine according to claim 26 to mammals.

29. The method according claim 28, wherein said administering is oral, intradermal, intramuscular, intraperitoneal, intravenous, subcutaneous, or intranasal.

30. A method of treating mammals infected with Mycobacterium tuberculosis comprising:
administering an effective amount of the antibody or binding portion thereof according to claim 13 to mammals infected with Mycobacterium tuberculosis.

31. The method according to claim 30, wherein said administering is oral, intradermal, intramuscular, intraperitoneal, intravenous, subcutaneous, or intranasal.

32. A composition for passively immunizing mammals infected with Mycobacterium tuberculosis comprising:
an isolated antibody or binding portion thereof according to claim 13; and
a pharmaceutically-acceptable carrier.

33. The composition according to claim 32, wherein the antibody is monoclonal or polyclonal.

34. A method of passively immunizing mammals infected with Mycobacterium tuberculosis comprising:
administering an effective amount of the composition according to claim 32 to mammals infected with Mycobacterium tuberculosis.

35. The method according to claim 34, wherein said administering is oral, intradermal, intramuscular, intraperitoneal, intravenous, subcutaneous, or intranasal.

36. A method of enhancing vaccination against Mycobacterium tuberculosis using a composition comprising a microorganism capable of producing an antigenic response against Mycobacterium tuberculosis when introduced into a host subject, said method comprising:
suppressing in the microorganism the expression of a nucleic acid molecule that either: 1) has a nucleotide sequence corresponding to SEQ ID NO: 1; 2) has a nucleotide sequence that hybridizes to the nucleic acid corresponding to SEQ ID NO: 1 under stringent conditions characterized by a hybridization buffer comprising 5×SSC at a temperature of 54° C.; 3) is at least 55% similar to the nucleotide sequence of SEQ ID NO: 1 by basic BLAST using default parameters analysis; or 4) encodes a protein or polypeptide having an amino acid sequence corresponding to SEQ ID NO: 2.

37. The method according to claim 36, wherein the microorganism is Mycobacterium bovis Bacillus Calmette-Guerin.

38. A composition for actively immunizing mammals against Mycobacterium tuberculosis comprising:
a microorganism capable of producing an antigenic response against Mycobacterium tuberculosis when introduced into a host subject, wherein the microorganism has been modified to be incapable of producing a nucleic acid molecule encoding a factor suppressing an immune response to Mycobacterium tuberculosis in a host, and
a pharmaceutically-acceptable carrier.

39. A composition according to claim 38, wherein the microorganism is Mycobacterium bovis Bacillus Calmette-Guerin.

40. A method of vaccinating a mammal against Mycobacterium tuberculosis comprising:
providing to a mammal an effective amount of the composition according to claim 38.

41. The method according to claim 38, wherein said administering is oral, intradermal, intramuscular, intraperitoneal, intravenous, subcutaneous, or intranasal.

42. A method of treating inflammatory disease in a mammal comprising:
providing a nucleic acid construct comprising:
a nucleic acid molecule that encodes a factor suppressing an immune response to Mycobacterium tuberculosis, wherein the nucleic acid molecule either: 1) has a nucleotide sequence corresponding to SEQ ID NO: 1; 2) has a nucleotide sequence that hybridizes to the nucleic acid corresponding to SEQ ID NO: 1 under stringent conditions characterized by a hybridization buffer comprising 5×SSC at a temperature of 54° C.; 3) is at least 55% similar to the nucleotide sequence of SEQ ID NO: 1 by basic BLAST using default parameters analysis; or 4) encodes a protein or polypeptide having an amino acid sequence corresponding to SEQ ID NO: 2; and
operably linked 5′ and 3′ regulatory elements; and
administering the nucleic acid construct to a mammal under conditions effective to treat an inflammatory disease.

43. The method according to claim 42, wherein the inflammatory disease is selected from the group consisting of bronchiectasis, asthma, sepsis, lupus, rheumatoid arthritis, scleroderma, inflammatory bowel diseases, multiple sclerosis, and tropical spastic paralysis.

44. The method according to claim 42, wherein the protein or polypeptide induces Il-10 production in the mammal.

45. A method of treating inflammatory disease in a mammal comprising:
providing a protein or polypeptide that suppresses an immune response to Mycobacterium tuberculosis, wherein the protein or polypeptide has an amino acid sequence of SEQ ID NO: 2; and
administering the protein or polypeptide to a mammal under conditions effective to treat an inflammatory disease.

46. The method according to claim 45, wherein the inflammatory disease is selected from the group consisting of bronchiectasis, asthma, sepsis, lupus, rheumatoid arthritis, scleroderma, inflammatory bowel diseases, multiple sclerosis, and tropical spastic paralysis.
